(19) United States
(12) Patent Application Publication
(10) Pub. No.: US 2014/0120543 A1
(43) Pub. Date: May 1, 2014

(54) EXPRESSION OF ISOFORM 202 OF ERCC1 FOR PREDICTING RESPONSE TO CANCER CHEMOTHERAPY

(71) Applicants: Ken Olaussen, Paris (FR); Jean-Charles Soria, Paris (FR); Luc Friboulet, Le Havre (FR)

(72) Inventors: Ken Olaussen, Paris (FR); Jean-Charles Soria, Paris (FR); Luc Friboulet, Le Havre (FR)

(73) Assignee: INSTITUTE GUSTAVE-ROUSSY, Villejuif (FR)

(21) Appl. No.: 14/021,316

(22) Filed: Sep. 9, 2013

Related U.S. Application Data
(63) Continuation-in-part of application No. 12/282,522, filed on Dec. 18, 2008.
Application No. 12/282,522 is a continuation-in-part of application No. 13/256,814, filed on Sep. 15, 2011.
Application No. 13/256,814 is a continuation-in-part of application No. 13/386,474, filed on Jan. 23, 2012, now abandoned.

(30) Foreign Application Priority Data
Mar. 14, 2006 (EP) 06290407.3

(51) Int. Cl. C12Q 1/68; G01N 33/573

(52) U.S. Cl. CPC C12Q 1/6886; G01N 33/573
USPC 435/6.12; 435/7.4

(57) ABSTRACT

An in vitro method for detecting the susceptibility of a tumor cell to a chemotherapy is disclosed. The method includes the step of measuring the expression level of the isoform 202 of the ERCC1 protein.

### TECHNICAL FIELD OF THE INVENTION

The present invention is directed to the detection of the Excision Repair Cross-Complementation group 1 (ERCC1) enzyme, and more specifically of the isoform 202 of this enzyme, and its use in the detection of the susceptibility of a tumor cell to chemotherapy, especially to platinating agents-based cancer chemotherapy. The invention also concerns a kit for detection, carrying out the method.

### BACKGROUND OF THE INVENTION

Lung cancer is a leading cause of cancer deaths in most industrialized countries (Jemal A, Murray T, Ward E, et al. Cancer statistics, 2005). Despite complete tumor resection in patients with stage I-III non-small-cell lung cancer, distant metastases develop in 50-70 percent of patients.

Adjuvant chemotherapy has been tested to improve survival in patients with completely resected non-small-cell lung cancer. The recently reported International Adjuvant Lung Cancer Trial (IALT) with 1,867 patients, was designed to assess the potential benefit of adjuvant cisplatin-based chemotherapy after complete resection of non-small cell lung cancer. The IALT demonstrated a 4.1 percent absolute benefit in 5-year overall survival in non-small-cell lung cancer patients treated with adjuvant cisplatin-based chemotherapy (the International Adjuvant Lung Cancer Trial Collaborative Group. Cisplatin-based adjuvant chemotherapy in patients with completely resected non-small-cell lung cancer. N Engl J Med 2004). Several other randomized studies have confirmed the benefit of postoperative platinum-based therapy in non-small-cell lung cancer (Gurubhagavatula S, Lynch T J. Semin Respir Crit. Care Med 2005). However, adjuvant chemotherapy only slightly prolongs survival, with a 5-year overall survival improvement ranging from 4 to 15 percent, and gives rise to significant adverse effects (Winton T, et al. N Engl J Med 2005). The identification and quantification of predictive factors for resistance or sensitivity to adjuvant cisplatin-based chemotherapy were therefore needed.

Among potential predictive factors are those involved in cisplatin-resistance such as DNA repair mechanisms. Cisplatin induces cytotoxic effects by binding to DNA and creating platinum-DNA adducts. Some of these adducts establish covalent cross-linking between DNA strands, thereby inhibiting DNA replication. Among the DNA repair pathways, nucleotide excision repair plays a central role and has been associated with resistance to platinum-based chemotherapy (Reed E. Cancer Treat Rev 1998). The excision repair cross-complementation group 1 (ERCC1) enzyme plays a rate-limiting role in the nucleotide excision repair pathway which recognizes and removes cisplatin-induced DNA adducts (Zamble D B. et al. Biochemistry 1996). ERCC1 is also an important factor in DNA interstrand cross-link repair, as well as in recombination processes (De Silva I U. et al. Mol Cell Biol 2000).

For more than a decade, smaller studies have repeatedly reported an association between low ERCC1 mRNA expression levels in several solid tumors and improved clinical outcomes in patients treated with platinum-containing regimens (Dabholkar M. et al J Clin Invest 1994). In particular, Lord et al (Lord R V et al. Clin Cancer Res 2002) reported that ERCC1 mRNA expression predicts response to chemotherapy in advanced non-small-cell lung cancer. Furthermore, by using methodologies such as DNA isolation, enzymatic digestions, and DNA sequencing, two common polymorphisms of the ERCC1 gene (codon 118 C/T and C8092A) were found to be correlated with response to platinum-based chemotherapy in colorectal (Viguier J. et al. Clin Cancer Res 2005) and non-small-cell lung cancer (Zhou W. et al. Clin Cancer Res 2004). These polymorphisms are mainly associated with lower translation rates of the ERCC1 gene, resulting in low expression levels.

The invention described in the international application WO 02/061128 (published on 8 Aug. 2002) relates to prognostic methods for cisplatin-based cancer chemotherapy assessing ERCC1 expression levels. These prognostic tests consist of (i) determining a platinum-based chemotherapy by examination of the amount of ERCC1 mRNA in patient's tumor cells and (ii) comparing it to a pre-determined threshold expression level. Such quantitative gene expression studies were developed for formalin-fixed paraffin-embedded pathological samples because most tumor samples are routinely formalin-fixed paraffin-embedded to allow histological analysis and subsequent archival storage. In this method, all the patients were treated with a platinum-based chemotherapy and the ERCC1 level was assessed so as to prognose the survival probability of the treated patients. Nevertheless, (i) techniques for the isolation and analyses of mRNA from formalin-fixed paraffin-embedded tissue samples are frequently inaccurate, costly and time-consuming and (ii) the conservation of mRNA in formalin-fixed paraffin-embedded samples is eventually affected with time. For these reasons, such analyses are carried out with difficulty by the skilled person, especially in large-scale studies. Other techniques, allowing an easier, more accurate and less expensive measure of the expression of ERCC1 are thus particularly needed.

Moreover, since the study on which the international application WO 02/061128 (published on 8 Aug. 2002) was based did not compare two groups of patients according to whether or not they were treated with cisplatin, the value of ERCC1 mRNA expression as evidenced in this study is only prognostic, and not predictive of the patient response to a chemotherapy.

A biomarker has a “prognostic value” if it enables to distinguish patients with high probability of survival from those who have low probability of survival, regardless of treatment or in a population where all patients received an identical treatment.

On another hand, a biomarker as a “predictive value” for a specific treatment if it enables to distinguish patients who have a high probability of clinical benefit (in terms of survival) from those who will take no benefit from said specific treatment. A predictive value of a biomarker can therefore only be demonstrated when two study groups are compared directly (a treated group against a non-treated group).

Furthermore, other studies have investigated the relation between the expression of different markers, like ERCC1, the platinum resistance and the prognosis in advanced non small cell lung cancer. Indeed, the scientific publication of Huang P Y et al. Chinese Journal of Cancer 2004 aims at determining prognostic values of different markers, like ERCC1, in response of a first-line platinum-based treatment. ERCC1 has been detected by immunohistochemistry. This study indicates that no prognostic value of ERCC1 expression can be demonstrated. On another hand, the publication of Watchers F M et al. Lung Cancer 2005 describes a study to determine a prognostic value of different protein expression involved in DNA repair. Among them, ERCC1 expression is measured in phase III-NSCLC patients by comparing first-line “cisplatin-gemcitabine” and “epirubicin-gemcitabine” chemotherapies. The ERCC1 expression was measured by immunohistochemistry on formalin-fixed, paraffin-embedded tumor biopsies. This document concludes that these markers (including ERCC1) are not prognostic of patient survival after these chemotherapies. Those two documents conclude that ERCC1 has no prognostic value of the efficiency of chemotherapy treatment.

### BRIEF SUMMARY OF THE INVENTION

These and other shortcomings of the prior art are addressed by the present invention, which provides an in vitro method for detecting the susceptibility of a tumor cell to a chemotherapy.

This invention differs from the anterior art, particularly the scientific publications of Huang P Y et al. Chinese Journal of Cancer 2004 and Watchers F M et al. Lung Cancer 2005 by the fact that the steps which have been carried out in said immunohistochemical method are different from those in these precedent documents wherein ERCC1 was not described as a predictive marker of a chemotherapy efficiency, but as a prognostic marker in patients treated by chemotherapy.

The method of the invention comprises the step of assessing the expression level of the isoform 202 of the ERCC1 protein. In a preferred embodiment of the invention, the said expression level is assessed by measuring the mRNA level of the isoform 202 of the ERCC1 protein, for example by RT-PCR or in situ hybridization. Alternatively, immunological methods aimed at measuring directly the protein level of the isoform 202 of ERCC1 (e.g., immunohistochemical or immunofluorescence assays) can be used.

Using an immunohistochemical assay, the inventors demonstrated, in a quantitative and reproducible way, that patients with ERCC1-negative tumors have a risk of death decreased by 33% (hazard ratio 0.67) when cisplatin based chemotherapy is added to surgery. On the other hand, the inventors have found that the risk of death was not decreased by the adjunction of chemotherapy among patients with ERCC1-positive tumors (hazard ratio 1.18) (Olaussen et al, NEJM, 2006). These results had been obtained with an anti-ERCC1 antibody recognizing, possibly among other isoforms, the isoform 202 of ERCC1.

It was stated in the patent application WO 02/061128 that protein expression levels could be only qualitatively monitored in formalin-fixed paraffin-embedded samples by using immunohistochemical methods. However, the present inventors have developed other quantitative and reproducible methods for assessing ERCC1 expression levels which is the subject matter of the present invention.

The analyses of the protein or mRNA level by the said methods are predictive of survival in early-stage and completely resected non-small cell lung cancer. Because immunological assays and mRNA analyses can be easily applied in every pathology laboratory, the present invention is therefore widely applicable and a useful test in clinical practice.

In a particular embodiment, the method of the invention also presents an additional advantage which is to be able to analyze formalin-fixed paraffin-embedded tumor samples, whatever the fixation techniques are.

The ERCC1 gene generates four isoforms (designated 201, 202, 203, and 204) by alternative splicing. As shown recently in Friboulet et al. (2013), using immunohistochemical analysis, the present Inventors showed that the level of biologic complexity of the ERCC1 protein had been underestimated and that the respective roles of the four ERCC1 protein isoforms had not been correctly assessed. In fact, they showed that the expression of nonfunctional ERCC1 isoforms leads to potential artifacts, with discrepant results.

More precisely, in this study, they demonstrated that only the reintroduction of the ERCC1-202 isoform rescued nucleotide excision repair activity and the capacity to repair cisplatin-induced DNA damage in established ERCC1-deficient cells. In terms of patient classification and therapeutic applications, these results suggested that evaluation of the expression of the unique functional isoform (ERCC1-202) constitutes a more accurate predictor of cisplatin benefit in patients with NSCLC than any other current approach. In other words, these data showed for the first time that therapeutic decision regarding cisplatin-containing treatment in patients with NSCLC requires the specific detection of the unique functional isoform of ERCC1-202.

These results explained the contradictory results obtained by the other teams so far, as none of them used antibodies that could distinguish functional ERCC1 isoform 202 from the other nonfunctional isoforms. Accordingly, it was difficult in the past to validate the correlation between the level of ERCC1 expression and overall survival on the basis of immunohistochemical detection, as abundant expression of one or several nonfunctional isoforms led to a false classification of the tumor as ERCC1-positive.

Using these unique ERCC1 deficient human NSCLC cell lines, the present Inventors further explored the influence of the different ERCC1 protein isoforms on DNA repair, protein-protein interactions and cellular mitotic process. Their data demonstrated that all currently known functions of ERCC1 are fulfilled by the same and unique ERCC1 isoform ERCC1-202 (see example 7 below).

Thus, the present invention provides an in vitro method for detecting the susceptibility of a tumor cell to a chemotherapy, said method comprising the step of measuring the expression level of the isoform 202 of the ERCC1 protein (hereafter referred to as “ERCC1-202”), for example by immunohistochemistry in a formalin-fixed paraffin-embedded tumor sample, by immunofluorescence on tumor cells or by mRNA analysis of tumor cell samples.

### BRIEF DESCRIPTION OF THE DRAWINGS

The subject matter that is regarded as the invention may be best understood by reference to the following description taken in conjunction with the accompanying drawing figures in which:

FIG. 1A shows an example of ERCC1 staining—Image (400*) of a strongly ERCC1-positive squamous cell carcinoma (Intensity=3);

FIG. 1B shows an example of ERCC1 staining—Image (400*) of an ERCC1-negative squamous cell carcinoma with positive internal controls (arrow);

FIG. 2A shows Kaplan-Meier estimates of the proportions of surviving patients—Overall survival according to treatment in all 761 patients—The adjusted hazard ratio for death in the chemotherapy group as compared with the control group was 0.87 (95 percent confidence interval, 0.71 to 1.06, P=0.17);

FIG. 2B shows Kaplan-Meier Estimates of the proportions of surviving patients—Overall survival according to treatment in patients with ERCC1-negative tumors—The adjusted hazard ratio for death in the chemotherapy group as compared with the control group was 0.67 (95 percent confidence interval, 0.51 to 0.89, P<0.006);

FIG. 2C shows Kaplan-Meier Estimates of the proportions of surviving patients—Disease-free survival according to treatment in patients with ERCC1-negative tumors. The hazard ratio for disease progression or death was 0.69 (95 percent confidence interval, 0.53 to 0.90, P<0.007);

FIG. 2D shows Kaplan-Meier Estimates of the proportions of surviving patients—Overall survival according to treatment in patients with ERCC1-positive tumors. The adjusted hazard ratio for death in the chemotherapy group as compared with the control group was 1.18 (95 percent confidence interval, 0.87 to 1.61, P=0.29);

FIG. 3A shows absence of negative dominant isoform—Western Blot detection of ERCC1 isoforms with 8F11 antibody—Cells expressing the 202 isoform were infected with lentivirus coding another ERCC1 isoform;

FIG. 3B shows absence of negative dominant isoform—IC50 assessment by WST-1 assay in cells treated for 48 hours with various concentrations of cisplatin;

FIG. 3C shows absence of negative dominant isoform—Clonogenic growth of isoform expressing cells treated two to three weeks with low cisplatin concentrations. Table specifies cisplatin IC50 (nM) values from clonogenic growth of cells;

FIG. 4 shows ERCC1 isoforms cellular localization—Western blot detection of ERCC1 protein isoforms after cellular fractionation;

FIG. 5A shows ERCC1 isoforms stability and expression—Western blot detection of ERCC1 protein isoforms after proteasome inhibition (MG132 at 2 μM for 24 h). An increase in ERCC1-201, -203 and -204 expression level suggested instability and degradation of these isoforms;

FIG. 5B shows ERCC1 isoforms stability and expression—Assessment by qRT-PCR of ERCC1 isoforms mRNAs in frozen samples from a series of 123 cases of resected NSCLC with matched tumour and normal specimens. The expression of ERCC1 isoform mRNA was determined using the 2-ΔΔCt method and data are presented as the fold-change in ERCC1 isoform mRNA expression relative to total ERCC1 mRNA. ERCC1-201 mRNA isoform was upregulated in tumours samples;

FIG. 6A shows ERCC1-isoform 202 is essential for proper chromosome segregation—Percentage of cells with nucleus size superior of mean of WT nucleus size determined on Diff Quick™ stain cells with ImageJ software;

FIG. 6B shows ERCC1-isoform 202 is essential for proper chromosome segregation—Percentage of multinucleated cells determined on Diff Quick™ stain cells manually counted (n=200);

FIG. 6C shows ERCC1-isoform 202 is essential for proper chromosome segregation—DNA content in cells blocked in G2/M cell cycle phase with Karyomax colcemid solution at 0.1 μg/ml for 6 h. Percentages of cells with high DNA content are shown;

FIG. 6D shows ERCC1-isoform 202 is essential for proper chromosome segregation—ERCC1 WT, attenuated and rescued cells. Centrosomes (-tubulin) were manually counted (n=100). Percentages of cells with more than 2 centrosomes are shown;

FIG. 6E shows ERCC1-isoform 202 is essential for proper chromosome segregation—E) 48 hours proliferation index in untreated ERCC1 WT, attenuated and rescued cells determined by WST-1 assay;

FIG. 7 shows ERCC1 isoforms function in ICL-R—IC50 assessment by WST-1 assay in cells treated for 48 h with various concentrations of the DNA interstrand cross-linking agent mitomycin-C. MMC IC50 (μM) values are indicated for each clone. Only 202 isoform restored MMC resistance compared to WT cells;

FIG. 8A shows ERCC1-protein complexes required ERCC1-202 isoform expression—Western blot detection of ERCC1 protein isoforms and ERCC1 interacting proteins;

FIG. 8B shows ERCC1-protein complexes required ERCC1-202 isoform expression—Proximity ligation assay (PLA, Duolink) detection of ERCC1/XPF heterodimers in A549 WT and ERCC1 deficient clone 216 expressing each of the four ERCC1 isoforms; and

FIG. 8C shows ERCC1-protein complexes required ERCC1-202 isoform expression—Proximity ligation assay (PLA-Duolink) detection of ERCC1/XPF, ERCC1/XPA, ERCC1/MSH2, ERCC1/FANCG, ERCC1/SLX4, ERCC1/Eg5, ERCC1/MAD2A, ERCC1/SLX4 and ERCC1/TRF2 heterodimers definite the unique binding ability of ERCC1-202 isoform only. ERCC1 was detected using FL297 antibody.

### DETAILED DESCRIPTION OF THE INVENTION

### ERCC1 Isoforms

ERCC1 was the first mammalian DNA repair gene to be cloned (Westerveld et al., 1984). ERCC1 gene contains 10 exons and codes for a pre-mRNA which leads by alternative splicing to four different isoforms (201, 202, 203, and 204). Very few data are available concerning the expression and role of the different ERCC1 isoforms.

The ERCC1-202 isoform is easily detectable. In human, the amino acid sequence of the ERCC1-202 isoform is for example the SEQ ID NO:2 (corresponding to the NCBI number NP_{—}001974.1). It is encoded for example by the mRNA sequence SEQ ID NO:1 (NM_{—}001983.3).

The alternatively spliced ERCC1-203 isoform, lacking the 72 bp of exon VIII, was first identified in 1986 (van Duin et al.). Van Duin et al. already suggested this isoform to be non-functional to repair ultraviolet (UV) light (NER) and mitomycin-C (interstrand cross-link repair—ICL-R) damages. Later, one report suggested this transcript may have a helper function for DNA repair of UV and mitomycin-C damages (Belt, 1991). In ovarian cancer tissues, highly variable splicing of ERCC1 mRNA was observed, and the alternatively spliced ERCC1-203 varied between 2 to 71% of the total ERCC1 mRNA (Dabholkar, 1994). Interestingly, the mRNA levels of this isoform were strongly inversely related to DNA repair activity, suggesting this shorter isoform to be a negative dominant of ERCC1 DNA repair function (Dabholkar, 1995; Yu, 1998). In small cell lung cancer cell line the ERCC1-203 protein isoform was found upregulated (Stordal 2009) after cisplatin exposure. This upregulation did not seem to have an influence on DNA repair efficiency in this study but the authors proposed this isoform to have a role in cell cycle arrest. Lately, ERCC1-203 isoform was confirmed to negatively affect the NER function of ERCC1 for cisplatin resistance in ovarian cancer cells (Sun et al, 2009). In human, the amino acid sequence of the ERCC1-203 isoform is for example the SEQ ID NO:6 (NP_{—}001159521.1). It is encoded for example by the mRNA sequence SEQ ID NO:5 (NM_{—}001166049.1).

First in mouse keratinocytes (Song et al., 2011), then in human malignant melanoma cells (Li W and Melton D W, 2011), the ERCC1-201 protein isoform was identified to be encoded by a larger ERCC1 transcript, originated from an upstream promoter. This transcript was found highly upregulated after cisplatin exposure by MAPK pathway in human melanoma cells (Li W and Melton D W, 2011). No functional analysis was accomplished about ERCC1-201 isoform but mutational analysis achieved by Sijbers et al. (1996) suggested this isoform to be non-functional. Indeed, they identified that the 91 N-terminal amino acids of ERCC1-202 isoform are dispensable for repair function, in contrast to a deletion of only four residues from the C-terminus. Since the ERCC1-201 isoform harbours an alternative C-terminus peptide sequence it is more than probable that it is non-functional with respect to NER activity. In human, the amino acid sequence of the ERCC1-201 isoform is for example the SEQ ID NO:4 (NP_{—}973730.1). It is encoded for example by the mRNA sequence SEQ ID NO:3 (NM_{—}202001.2).

The ERCC1-204 isoform, which lack the long 215 bp exon-3, has never been reported in the literature. This isoform still have the XPF, MSH2 and dsDNA binding domains but lack a part of the XPA and ssDNA binding domains. Thus this isoform could negatively influence DNA repair by sequestration of XPF or MSH2. In human, the amino acid sequence of the ERCC1-204 isoform is for example the SEQ ID NO:8 (NSP00000394875.2). It is encoded for example by the mRNA sequence SEQ ID NO:7 (ENST00000423698.2).

### The Method of the Invention

The present invention provides in vitro methods for detecting the susceptibility of a tumor cell to a chemotherapy, said method comprising the step of measuring the expression level of the isoform 202 of the ERCC1 protein (ERCC1-202).

This expression level can be assessed either by measuring the mRNA level (e.g., by RT-PCR or by in situ hybridization or Duolink) or by measuring the protein level of this isoform, for example by means of an immunological method.

By “immunological method”, it is herein meant any experimental method involving antibodies that are able to recognize specifically the isoform 202 of the ERCC1 protein. These immunological methods can be in particular an immunohistochemistry assay or an immunofluorescence assay.

In an “immunohistochemical assay”, a section of tissue is tested for specific proteins by exposing the tissue to antibodies that are specific for the protein that is being assayed. The antibodies are then visualized by any of a number of methods to determine the presence and amount of the protein present. Examples of methods used to visualize antibodies are, for example, through enzymes linked to the antibodies (e.g., luciferase, alkaline phosphatase, horseradish peroxidase, or P-galactosidase), or chemical methods (e.g., DAB/Substrate chromagen), gold, fluorescent or labelled antibodies by any of the many different methods known to those skilled in this art. In embodiments of the said immunohistochemical method, detection or assaying the level of the isoform 202 of the ERCC1 protein in a tumor sample includes contacting it with an antibody or antigen-binding fragments directed against said isoform or fragments thereof; and determining the amount of the binding antibody on the tumor sample.

In contrast thereto, as used herein, an “immunofluorescence assay” is a technique using fluorescence microscopy on cells that have been extracted from the tumor tissue or on cell lines, e.g., on cultured cell lines or on individual cells. This technique uses the specificity of antibodies to their antigen to target fluorescent dyes to specific biomolecule targets within a cell, and therefore allows visualization of the distribution of the target molecule (the isoform 202 of ERCC1) through the sample. Immunofluorescence can be used in combination with other, non-antibody methods of fluorescent staining, for example, use of DAPI to label DNA.

Several microscope designs can be used for immunohistochemical or immunofluorescence assays; the simplest is the epifluorescence microscope. The confocal microscope is also widely used. Various super-resolution microscope systems that are capable of much higher resolution can also be used.

As used herein, the term “antibody” includes immunoglobulin molecules and antigen binding fragments thereof. The antibody can be a polyclonal antibody or a monoclonal antibody. The antibody can be labeled by a detectable means and includes enzymatically, radioactively, fluorescently, chemiluminescently or bioluminescently labeled antibodies by any of the many different methods known to those skilled in this art.

By “antigen-binding fragments” it is intended to encompassed particularly the fragments Fv, Fab, F(ab′)2, Fab′, scFv, scFv-Fc. These antibody fragments are obtained using conventional techniques well-known to those with skill in the art, and the fragments are screened for utility in the same manner as are intact antibodies.

In the context of the present invention, an antibody is said to “recognize” or “bind” the ERCC1 isoform 202 of SEQ ID NO:2 if said antibody has an affinity constant Kₐ (which is the inverted dissociation constant, i.e. 1/K_{d}) higher than 10⁷ M⁻¹, preferably higher than 10⁸ M⁻¹, more preferably higher than 10⁹ M⁻¹ for said isoform. Also, in the context of the present invention, an antibody is said to “specifically bind” or to “specifically recognize” the ERCC1 isoform 202 of SEQ ID NO:2 if said antibody has an affinity constant Kₐ higher than 10⁷ M⁻¹, preferably higher than 10⁸ M⁻¹, more preferably higher than 10⁹ M⁻¹ for said isoform and has an affinity constant Kₐ lower than 10⁵ M⁻¹ for all the other proteins, including the other isoforms of ERCC1.

The affinity constant which is used to characterize the binding of antibodies (Ab) to a peptide or an antigen (Ag) is the inverted dissociation constant defined as follows:

$$Ab + Ag ⇌ AbAg$$

$${K}_{a} = \frac{[ AbAg ]}{[ Ab ] [ Ag ]} = \frac{1}{{K}_{d}}$$

This affinity can be measured for example by equilibrium dialysis or by fluorescence quenching, both technologies being routinely used in the art.

In a preferred embodiment, the antibody of the invention binds the ERCC1 isoform 202 of SEQ ID NO:2 with a K_{D} of less than 10⁻⁹M, preferably of less than 10⁻¹⁰ M.

The present inventors have shown that only the 202 isoform of ERCC1 can form stable heterodimer complexes with the DNA repair endonuclease XPF (SEQ ID NO: 9, corresponding to NP_{—}005227.1, which is encoded by the ERCC4 gene), with the XPA protein (SEQ ID NO:10, corresponding to NP_{—}000371.1), with the DNA mismatch repair protein MSH2 (SEQ ID NO: 11 corresponding to the isoform 1 (full length) NP_{—}000242.1 or SEQ ID NO: 12 corresponding to the isoform 2 NP_{—}001245210.1), with the Fanconi anemia Group G protein FANC G (SEQ ID NO: 13, corresponding to NP_{—}004620.1), with the SLX4 protein (SEQ ID NO:14 corresponding to NP_{—}115820.2), with the kinesin Eg5 (SEQ ID NO: 15, corresponding to NP_{—}004514.2 encoded by the KIF11 gene), with the MAD2A protein (SEQ ID NO: 16, corresponding to NP_{—}002349.1 encoded by the MAD2L1 gene), and the protein TRF2 (SEQ ID NO: 17, corresponding to NP_{—}005643.2, encoded by the TERF2 gene).

Their data clearly suggest that detecting the presence of ERCC1/XPF, ERCC1/XPA, ERCC1/MSH2, ERCC1/FANCG, ERCC1/SLX4, ERCC1/Eg5, ERCC1/MAD2A, or of ERCC1/TRF2 heterodimers is helpful for quantifying the level of functional ERCC1-202 isoform in cancer cell.

Consequently, the detecting method of the invention may comprise the step of detecting and/or quantifying the presence of a stable heterodimers selected from the group consisting of: ERCC1/XPF, ERCC1/XPA, ERCC1/MSH2, ERCC1/FANCG, ERCC1/SLX4, ERCC1/Eg5, ERCC1/MAD2A, and ERCC1/TRF2. This detection/quantification may be performed by conventional means, such as protein complex immunoprecipitation, pull-down assays, Proximity ligation assay (PLA), FRET assays, surface plasmon resonance (SPR) assays, affinity capture mass spectrometry or the Duolink® immunoassay developed by Olink. Immunoassays such as immunoprecipitation, pull-down assays and the Duolink® assay are herein preferred. Proximity ligation assay (PLA) is even more preferred.

In one embodiment, the skilled person will preferably use an antibody which binds specifically to the XPF protein, for example the anti-XPF antibody clone 3F2/3; or an antibody which binds specifically to the XPA protein, for example the anti-XPA antibody clone 5A2 commercialized by Pierce under the reference “MA1-21460”; or an antibody which binds specifically to the MSH2 protein, for example the anti-MSH2 antibody commercialized by BIORBYT under the reference “orb16010”; or an antibody which binds specifically to the FANCG protein, for example the anti-FANCG antibody commercialized by Abcam under the reference “ab54645”; or an antibody which binds specifically to the Eg5 protein, for example the anti-Eg5 antibody commercialized by Abcam under the reference “ab51976”; or an antibody which binds specifically to the SLX4 protein, for example the anti-SLX4 antibody commercialized by Abnova under the reference “H00084464”; or an antibody which binds specifically to the MAD2A protein, for example the anti-MAD2A antibody clone 17D10 commercialized by Abcam under the reference “10691”; or an antibody which binds specifically to the TRF2 protein, for example the anti-TRF2 antibody clone 4A794 commercialized by Abcam under the reference “ab13579”.

In another embodiment, the skilled person will preferably use an antibody which binds specifically to the ERCC1-XPF heterodimer, the ERCC1/XPA heterodimer, the ERCC1/MSH2 heterodimer, the ERCC1/FANCG heterodimer, the ERCC1/SLX4 heterodimer, the ERCC1/Eg5 heterodimer, the ERCC1/MAD2A heterodimer, and the ERCC1/TRF2 heterodimer.

In the method of the present invention, the antibody recognizing ERCC1-202 is preferably selected in the group consisting of: the mouse monoclonal antibody clone 8F1 (Thermo Scientific Inc. ERCC1 Ab-2 Cat. MS-671-P1), the mouse monoclonal antibody 3H11 (Novus Biologicals Cat. NB100-117 or Santa Cruz Biotechnology Inc. Cat. sc53281), the rabbit polyclonal antibody FL297 (Santa Cruz Biotechnology Inc. Cat. sc-10785), the rabbit monoclonal antibody EP2143Y (Origene Inc. Cat. TA306972), the mouse monoclonal antibody 4F9 (Origene. Inc. Cat. UM500008), and the mouse monoclonal antibody 2E12 (Origene. Inc. Cat. UM500011).

Interestingly, the method of the present invention can be carried out on post-operative patient tumor samples. The chemotherapy will then be applied after a surgical resection of the tumor.

In a preferred embodiment of the invention, the in vitro method of the invention is thus for detecting susceptibility to a chemotherapy of a tumor cell from patients who had undergone a surgical resection of their tumor.

The method of the invention enables to predict if a platinum-based chemotherapy will be of beneficial use in patients suffering from cancer. As a matter of fact, the present inventors demonstrated that the expression level of ERCC1-202 impacts the efficiency of a platinum-based chemotherapy treatment. In particular, their results suggest that, when the expression level of ERCC1-202 is low, then platinum-based chemotherapy will be efficient and said patient will experience long survival upon treatment with this platinum-based chemotherapy. Conversely, when the expression level of ERCC1-202 is high, then a platinum-based chemotherapy will be useless because the patient's survival will not increase upon treatment with this platinum-based chemotherapy. In this case, a platinum-based chemotherapy is to be avoided and another treatment is to be preferred (for example, surgery, immunotherapy, radiotherapy, platinum-free chemotherapy, etc.).

Thus, in another aspect, the present invention relates to a method for treating a patient suffering from cancer, containing the steps of:
i) detecting the susceptibility of the tumor cells of said patient to a chemotherapy by means of the measuring the ERCC1-202 expression level as disclosed above, and
ii) if said tumor cells express low level of ERCC1-202, then administering to said patient an efficient dose of a platinum-based chemotherapy, whereas
if said tumor cells express high level of ERCC1-202, then treating said patient with a platinum-free chemotherapy, by surgery, by radiotherapy, or by immunotherapy.

Determining if the expression level of ERCC1-202 is low or high in a tumor sample can be performed by comparing the expression level of ERCC1-202 in said sample with the expression level of ERCC1-202 obtained in an internal positive control which is used as a reference. Immunostaining intensity is for example multiplied by a proportion score (representative of the percentage of positive tumor nuclei) to obtain a final quantitative H-Score (for “Histology-score”). The median value of the H-Scores was a priori chosen as the cut-off point.

In immunohistochemical assays, high levels of ERCC1-202 are for example detected when the H Score exceeding median value of H-Score (i.e., tumors with a staining intensity score of 2 and with 50% or more positive nuclei or with a staining intensity score of 3 and 10% or more positive nuclei). Low levels of ERCC1-202 are detected for example when the H-Score is below the median value of H-Score. In Duolink assays, expression level of ERCC1-202 is obtained by assessing the number of fluorescent points in the nuclei of tumoral cells.

Preferably, internal positive control herein consists of stroma cells surrounding the tumor area.

In a preferred embodiment of the invention, the method is based on an immunohistochemical assay comprising the following steps:
(a) obtaining slides from formalin-fixed paraffin-embedded tumor samples,
(b) retrieving epitope in buffer,
(c) incubating slides with a monoclonal ERCC1 antibody recognizing specifically the isoform 202 of ERCC1,
(d) determining the amount of binding antibodies on the formalin-fixed paraffin-embedded tumor samples, using the amount of binding antibodies on an internal positive control as a reference,
(e) determining the percentage of labeled nuclei on the formalin-fixed paraffin-embedded tumor samples,
(f) multiplying the value estimated in step (d) with the value estimated in step (e), and
(g) determining a platinum-based chemotherapy regimen by comparing the value obtained in step (f) to a pre-determined threshold level.

With this method, steps (d) and (f) are used for the first time. They make the detection of ERCC1 surprisingly quantitative and reproducible.

In a more preferred embodiment of the invention, such cancer is preferably a non-small-cell lung cancer.

In another preferred embodiment of the invention, the cancer chemotherapy is a platinum-based cancer chemotherapy.

It is also preferred that the cancer chemotherapy is based on cisplatin alone or associated with other chemotherapeutic agents as etoposide or a vinca alkaloid.

The invention also relates to a kit for the detection or quantification of the isoform 202 of the ERCC1 protein (ERCC1-202), wherein said kit comprises antibodies and appropriate reagents and buffers. The antibody used in this kit is the monoclonal ERCC1 mouse antibody clone 8F1 commercialized by Neomarkers, the mouse monoclonal antibody 3H11 (distributed by several manufacturers), the rabbit polyclonal antibody FL297 (Santa Cruz), or the rabbit monoclonal antibody EP2143Y (distributed by several manufacturers).

In a preferred embodiment, said detection kit is used for detecting the susceptibility of a tumor cell to a chemotherapy, for example in the method described above, or in a method for treating a patient suffering from cancer, for example in the method described above.

### EXAMPLES

### Example 1

### Materials and Methods

### Patients and Study Design.

All patients had participated in the IALT study that compared adjuvant cisplatin-based chemotherapy to observation in patients with non-small-cell lung cancer. Inclusion criteria and the results of the IALT have already been reported (The International Adjuvant Lung Cancer Trial Collaborative Group. Cisplatin-based adjuvant chemotherapy in patients with completely resected non-small-cell lung cancer. N Engl J Med 2004; 350:351-60), see table 1. Briefly, 1,867 patients with completely resected stage I-III non-small-cell lung cancer had been randomized to either chemotherapy with cisplatin (total dose 300-400 mg/m<2>) plus another drug (etoposide or a vinca alkaloid), or observation (control group). The median follow-up time was 56 months.

The IALT-Bio study was subsequently designed by a steering committee to examine whether immunohistochemically assessed tumor markers had the ability to predict a survival benefit from chemotherapy in formalin-fixed paraffin-embedded tumor samples collected from centers that had recruited more than 10 patients. To study whether the effect of chemotherapy was different between patients with a positive or a negative marker status, the estimated power to detect a 20 percent difference in the survival benefit at 5 years in 800 patients was 66 percent (two-sided, type I error 1%). Twenty-eight centers in 14 countries (see table 1) contributed specimens.

Approval was obtained from the local Institutional Review Boards according to the legal regulations in each participating country.

All tumors were reviewed centrally (Brambilla E, Lantuejoul S, Dunant A, et al. IALT—International Adjuvant Lung Cancer Trial—: Quality assessment and histopathological review according to the WHO 2004 classification and assessment of prognostic and predictive role of pathological criteria. Lung Cancer 2005; 49 Suppl. 2:S44) according to the W.H.O. 2004 histo-pathological classification.

TABLE 1
The IALT-Bio participating centers (investigators and pathologists)

| | |
|---|---|
| AUSTRIA: | R. Pirker, Internal Medicine I, Vienna, G. Dekan, Institute of |
| | Pathology, Vienna |
| BELGIUM: | J. Vansteenkiste, University Hospital, Leuven |
| BRAZIL: | I. Sathler Pinel, Instituto Nacional de Cancer, Rio de Janeiro |
| | R. Younes, Hospital A.C. Camarco, Sao Paulo |
| FRANCE: | A.A. Kanoui, Centre Physiothérapie du Rouget, Sarcelles; |
| | R. Dachez, Laboratoire L.C.L., Paris; |
| | S. Deslignères, Hospital Delafontaine, Saint-Denis; |
| | O. Languille-Mimoune, Cabinet Pathologie, Paris; |
| | P. Sabatier, Centre Hospitalier Victor Dupouy, Argenteuil |
| | T. Le Chevalier, Institut Gustave-Roussy, Villejuif; |
| | M. Antoine, Hôpital Tenon, Paris |
| | P. Boz, Cabinet de Pathologie, Papeete; |
| | P. Bruneval, Association Promotion Anatomie Pathologique, Paris; |
| | M.C. Charpentier, Cabinet Pathologie Tolbiac, Paris; |
| | B. Chetaille, Hôpital Sainte Marguerite, Marseille; |
| | E. Dulmet, Centre Chirurgical Marie-Lannelongue, Le Plessis Robinson; |
| | F. Capron, Groupe Hospitalier Pitié-Salpétrière, Paris; |
| | B. Gosselin, C.H.U., Lille; |
| | D. Grunenwald, P. Validire, Institut Mutualiste Montsouris, Paris; |
| | F. Labrousse, C.H.U., Limoges; |
| | N. Pericoli, Roma (Italy); |
| | D. Petrot, Cabinet d'Anatomie Pathologique, Niort; |
| | N. Rouyer, Cabinet de Pathologie Butet-Rouyer, Nice |
| | B. Milleron, M. Antoine, Hôpital Tenon, Paris |
| | J.F. Morère, M. Kambouchner, Hôpital Avicenne, Bobigny |
| | G. Ozenne, Ceditrac-CMC du Cèdre, Bois Guillaume |
| | T. Ducastelle, Laboratoire d'Anatomie et Cytologie, Rouen |
| | E. Quoix, Hôpital Lyautey, Strasbourg; |
| | P. Durand de Grossouvre, Laboratoire d'Anatomie Pathologique, |
| | Haguenau; |
| | B. Gasser, C.H.U., Strasbourg |
| | A. Rivière, Centre François Baclesse, Caen; |
| | F. Galateau-Salle, CHU, Caen |
| | C. Tuchais, P. Jallet, G. Bertrand, I. Valo, Centre Paul Papin, |
| | Angers |
| GERMANY: | W. Eberhardt, University Hospital, Essen; |
| | D. Theegarten, Institute of Pathology, Ruhr-University Bochum, Bochum |
| GREECE: | P. Christaki, Papanikolaou General Hospital, Pylea |
| | T. Dosios, V. Kyriakou, Athens University School of Medicine, |
| | Athens |
| | E. Papadakis, P. Agelidou, Sotiria Hospital, Athens |
| | K. Zarogoulidis, University Hospital, Thessaloniki |
| ITALY: | A. Masotti, Azienda Ospedaliera Di Verona, Verona |
| LITHUANIA: | A. Jackevicius, Institute of Oncology Vilnius University, Vilnius |
| POLAND: | J. Laudanski, L. Chyczewski, M. Kozlowski, J. Niklinski, Medical |
| | School, Bialystok |
| | T. Grodski, J. Pankowski, Regional Hosp. For Lung Diseases, |
| | Szczecin |
| | T. Orlowski, M. Chabowski, R. Langfort, Institute of Tuberculosis |
| | and Lung Disease, Warsaw; |
| | B. Muszczynska-Bernhard, Dolnoslaskiego Centrum Chorob Pluc, |
| | Wroclaw |
| ROMANIA: | T. Ciuleanu, Oncological Institute ″Ion Chiricuta″, Cluj-Napoca |
| SLOVAKIA: | J. Baumohl, University Teach. Hospital, Kosice |
| SPAIN: | F. Cardenal, Hospital Duran I Reynals, Barcelona; R. Bernat, |
| | Hospital de Bellvitge, Barcelona |
| | J. Salinas, J.B. Lopez, Hospital Virgen de Arrixaca, EI Palmar |
| | Murcia |
| SWEDEN: | B. Bergman, A. Hussein, Sahlgrenska Hospital, Göteborg |
| YUGOSLAVIA: | G. Radosavljevic, Institute for Lung Disease, Belgrade |

### Immunostaining for ERCC1.

The epitopes were first retrieved in citrate buffer (10 mM, pH 6.0, heated for 30 minutes in a bain marie), then slides were incubated at a 1:300 dilution over 60 minutes with the monoclonal ERCC1 mouse antibody (clone 8F1, NeoMarkers, Fremont Calif., USA) that was raised against the full-length human ERCC1 protein. Antibody binding was detected by means of an ABC-kit with NovaRED™ as the substrate (Vectastain Elite, Vector Laboratories, Burlingame Calif., USA) and Mayer's hematoxylin as the counterstain. Sections of normal tonsil tissues were included as external positive controls and stromal cells (endothelium) surrounding the tumor area served as internal positive controls.

### Microscopic Analysis

Two investigators who where blinded to clinical data, independently evaluated ERCC1 staining under the light microscope at *400 magnification. We recorded whether or not tumor or stromal cells expressed ERCC1. In addition, staining intensity was graded on a scale of 0 to 3 (using endothelial cells in tonsil controls as a reference point [intensity 2]). Discordant cases were reviewed. Cases without valid internal controls were excluded. Five images of representative areas were acquired at *400 magnification for each case. All positive or negative tumor nuclei (a total of 500-1,500 tumor nuclei per case) were manually counted on a computer screen using ImageJ freeware edited by the National Institutes of Health (http://rsb.info.nih.gov/ij). The percentage of positive tumor nuclei was calculated per case and a proportion score was attributed (0 if 0 percent; 0.1 if 1 to 9 percent; 0.5 if 10 to 49 percent; 1.0 if 50 percent or more), as previously described (Al Haddad S, Zhang Z, Leygue E, et al. Psoriasin (S100A7) expression and invasive breast cancer. Am J Pathol 1999; 155:2057-66 or Handra-Luca A, Bilal H, Bertrand J C, Fouret P. Extra-cellular signal-regulated ERK-1/ERK-2 pathway activation in human salivary gland mucoepidermoid carcinoma: association to aggressive tumor behavior and tumor cell proliferation. Am J Pathol 2003; 163:957-67). In each case, the proportion score was multiplied by the staining intensity of nuclei to obtain a final quantitative H-score (among 9 possible ones). The median value of the H-scores was a priori chosen as the cut-off point for separating ERCC1-positive from ERCC1-negative tumors.

### Statistical Analysis.

As in the IALT, the primary endpoint was overall survival after the date of randomization. Disease-free survival was analyzed as a secondary endpoint. In order to study selection bias within the IALT-Bio participating centers, the pre-randomization characteristics and overall survival of the two groups of patients (with or without blocks) were compared using a Cox model. Baseline data according to the ERCC1 status were compared in univariate analyses with Chi-square tests and with a multivariate logistic model.

Survival rates were estimated using the Kaplan-Meier method. The predictive values of the ERCC1 status and chemotherapy for survival were studied using the Cox model. As in the IALT analysis, the Cox model included every factor used in the stratified randomization (center, disease stage, and type of surgery), plus clinical and histological predictive factors (age, sex, W.H.O. performance status, and revised histopathological type). All other factors that were statistically related to the ERCC1 status in the multivariate logistic model (P<0.05) were added to the survival Cox model (pathological T status, and pleural invasion). The predictive value of ERCC1 was studied by testing the interaction between the ERCC1 status and the attributed treatment (chemotherapy or no chemotherapy) in the same Cox model. All reported P values were two-sided. P values below 0.01 were considered statistically significant in order to limit the risk of false positive results. All analyses were performed using SAS software, version 8.2.

### Example 2

### Patient Characteristics

The 28 centers which participated in the IALT-Bio study included 1045 patients in the original IALT study. They were able to provide one tumor block for only 867 patients (83 percent). These 867 patients were comparable to the remaining 178 in terms of pre-randomization characteristics and overall survival. The amount and quality of the 824 blocks were adequate for serial sectioning. Among these blocks, 783 contained tumor material corresponding to non-small-cell lung cancer and were included in the IALT-Bio study. After exclusion of cases without valid positive internal controls, ERCC1 expression was evaluated in 761 cases. All further statistical analyses were based on these 761 patients.

The characteristics of the IALT-Bio study patient population are summarized in Table 1. A total of 426 cases were squamous-cell carcinomas (56 percent), 242 adenocarcinomas (32 percent), and 93 were of another histological type (12 percent). Median age was 58 years (range 27-77) and the great majority were males (81.6 percent). Three hundred and eighty-nine patients (51 percent) were randomized to receive adjuvant cisplatin-based chemotherapy, whereas 372 (49 percent) were randomized to the control group.

### Example 3

### Immunohistochemically Assessed ERCC1 Expression

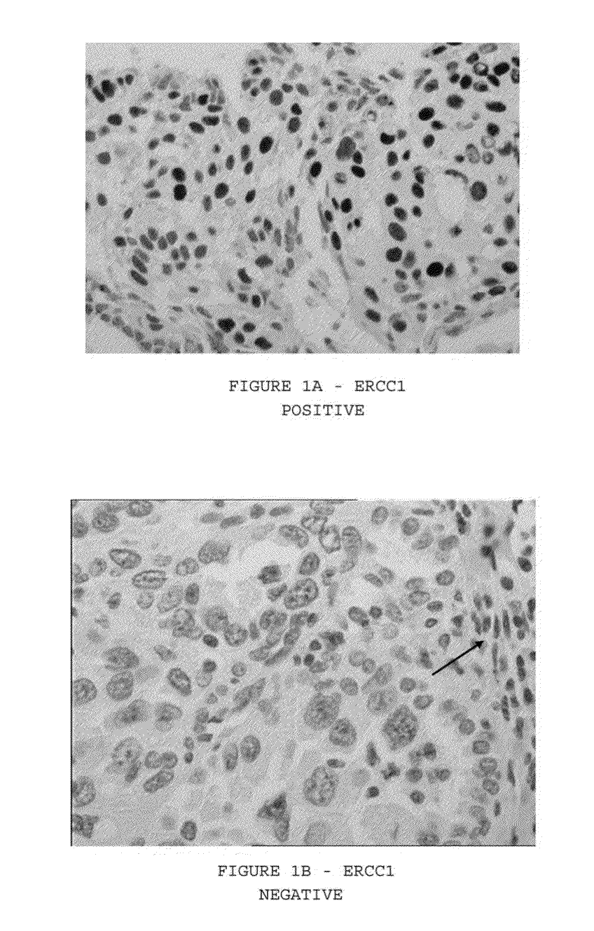

As illustrated in FIG. 1, ERCC1 immunostaining was nuclear. The median value of the percentage of stained cells was 24 percent (range 0 to 100 percent), whereas the median value of H-scores was 1.0 Tumors with an H-score exceeding 1.0 (i.e. tumors with a staining intensity score of 2 and 50 percent or more positive nuclei or a staining intensity score of 3 and 10 percent or more positive nuclei) were deemed ERCC1 positive, which was the case in 335 patients (44 percent). The median H-score alone (1.0) was attributed to 164 tumors (22 percent). The main differences in clinico-pathological parameters according to ERCC1 expression are reported in Table 2 (univariate analysis). Using the multivariate logistic model, ERCC1 expression was significantly correlated with age (P=0.02 lower in young patients), sex (P=0.04 lower in females), pathological T status (P=0.04 lower with a higher T status), histological type (lower in adenocarcinomas P<0.0001), and pleural invasion (P=0.01 higher in the case of pleural invasion).

TABLE 2
Patient Characteristics

| | Total | ERCC1+ | ERCC1− | |
|---|---|---|---|---|
| | N = 761 | N = 335 | N = 426 | |
| Characteristic | (percent) | (percent) | (percent) | P-value* |
| Age | | | | P < 0.003 |
| <55 yr | 231 (30) | 80 (24) | 151 (35) | (P for trend: |
| 55-64 yr | 330 (43) | 161 (48) | 169 (40) | P < 0.008) |
| >64 yr | 200 (26) | 94 (28) | 106 (25) | |
| Sex | | | | P < 0.0005 |
| Male | 621 (82) | 292 (87) | 329 (77) | |
| Female | 140 (18) | 43 (13) | 97 (23) | |
| Pathological TNM | | | | P = 0.97 |
| stage | | | | |
| Stage I | 267 (35) | 119 (35) | 148 (36) | |
| Stage II | 175 (23) | 76 (23) | 99 (23) | |
| Stage III | 319 (42) | 140 (42) | 179 (42) | |
| T of TNM | | | | P = 0.10 |
| 1 | 118 (16) | 60 (18) | 58 (14) | |
| 2 | 452 (59) | 188 (56) | 264 (62) | |
| 3 | 181 (24) | 85 (25) | 96 (23) | |
| 4 | 10 (1) | 2 (1) | 8 (2) | |
| Histological type | | | | P < 0.0001 |
| Squamous cell | 426 (56) | 236 (70) | 190 (45) | |
| carcinoma | | | | |
| Adenocarcinoma | 242 (32) | 71 (21) | 171 (40) | |
| Other | 93 (12) | 28 (8) | 65 (15) | |
| Performance Status | | | | P = 0.06 |
| 0 | 426 (56) | 188 (56) | 238 (56) | |
| 1 | 276 (36) | 113 (34) | 163 (38) | |
| 2 | 59 (8) | 34 (10) | 25 (6) | |
| Pleural invasion | | | | P < 0.007 |
| Yes | 61 (8) | 37 (11) | 24 (6) | |
| No | 700 (92) | 298 (89) | 402 (94) | |
| Vascular invasion | | | | P = 0.04 |
| Yes | 222 (29) | 85 (25) | 137 (32) | |
| No | 539 (71) | 250 (75) | 289 (68) | |
| Surgery | | | | P = 0.35 |
| Pneumonectomy | 306 (40) | 141 (42) | 165 (39) | |
| Segment-/lobectomy | 455 (60) | 194 (58) | 261 (61) | |
| Radiotherapy | | | | P = 0.35 |
| Yes | 199 (26) | 82 (24) | 117 (27) | |
| No | 562 (74) | 253 (76) | 309 (73) | |
| Planned cisplatin dose | | | | P = 0.67 |
| 80 mg/m² per cycle | 139 (18) | 58 (17) | 81 (19) | |
| 100 mg/m² per cycle | 544 (71) | 245 (73) | 299 (70) | |
| 120 mg/m² per cycle | 78 (10) | 32 (10) | 46 (11) | |

TABLE 3
Variation of overall survival according to attributed treatment and
ERCC1 status

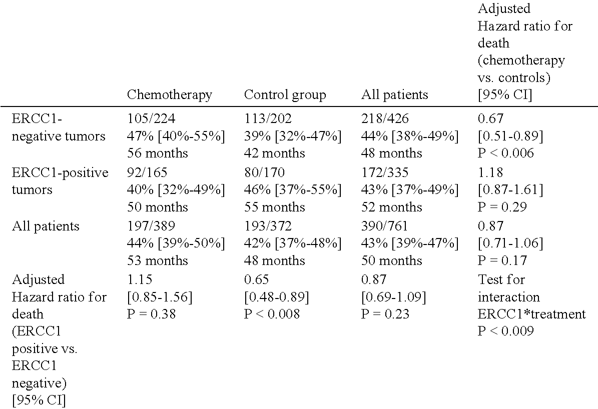

CI denotes confidence interval
the central grey [symbol] cells contain the following information: number of deaths, number of patients, 5-year survival rate and the 95 percent confidence interval, and median survival.

### Example 4

### Overall Survival and ERCC1 Expression

The 5-year overall survival rate was 43 percent, 95 percent confidence interval [39 to 47 percent] (Table 3) for the total study-population. Using the Cox model, ERCC1 expression had no predictive value for the entire study population (adjusted hazard ratio for death, 0.87; 95 percent confidence interval [0.69 to 1.09], P=0.23).

### Example 5

### Overall Survival and Adjuvant Chemotherapy

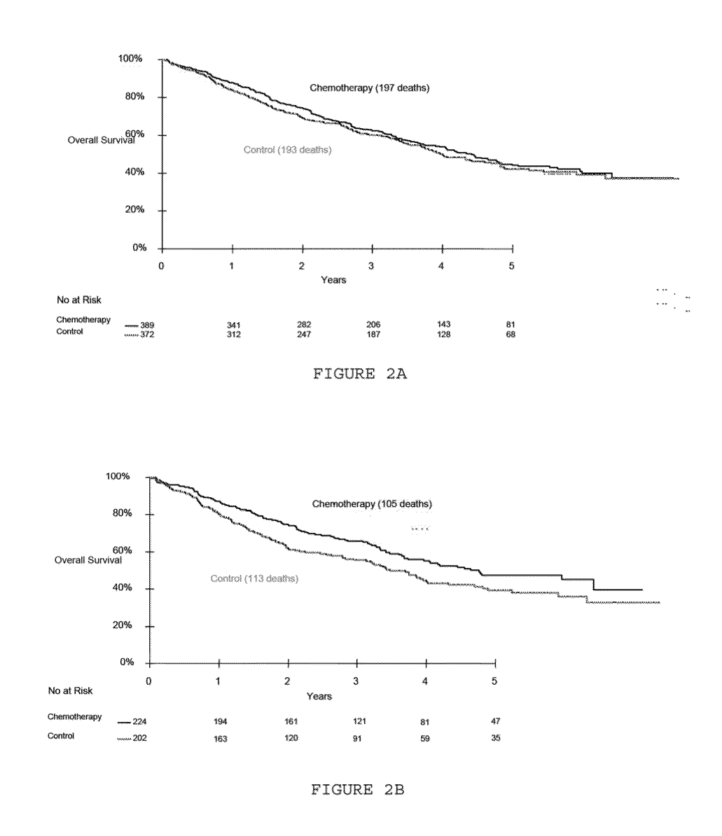

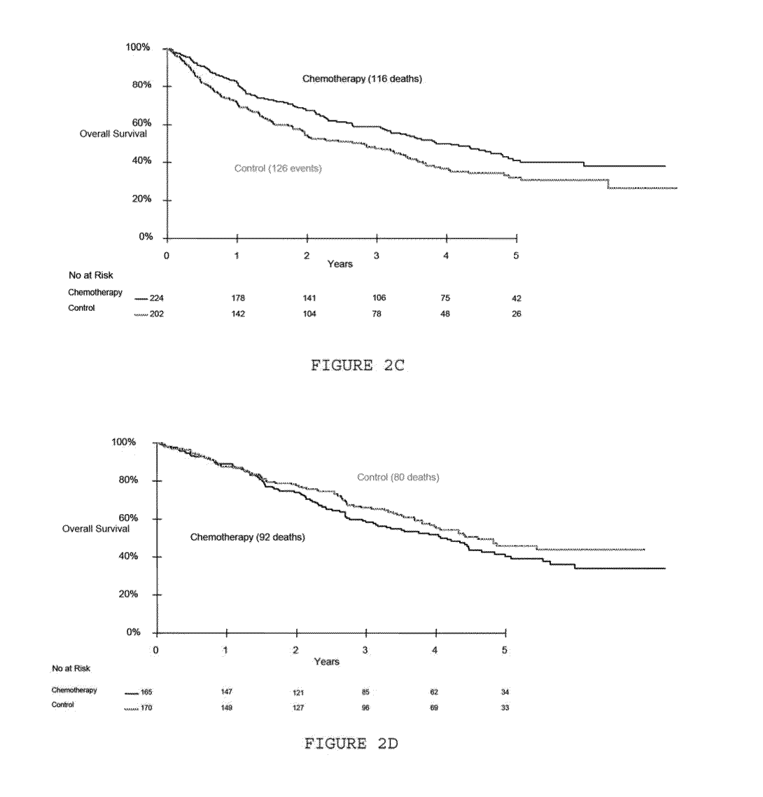

The 5-year overall survival rates were 44 percent (95 percent confidence interval [39 to 50 percent]) and 42 percent (95 percent confidence interval [37 to 48 percent]) in the chemotherapy group and control group respectively (Table 3). In the Cox model, the adjusted hazard ratio for death was 0.87 (95 percent confidence interval [0.71 to 1.06], P=0.17) in favor of chemotherapy (Table 3, FIG. 2A).

### Example 6

### Benefit of Adjuvant Chemotherapy According to ERCC1 Expression

The interaction term between ERCC1 expression and treatment was statistically significant (for overall survival, P<0.009). In patients with ERCC1-negative tumors, overall survival was significantly higher in the chemotherapy group compared to the control group (adjusted hazard ratio for death, 0.67; 95 percent confidence interval [0.51 to 0.89] P<0.006) (Table 3). The 5-year survival rates were 47 percent (95 percent confidence interval [40 to 55 percent]) and 39 percent (95 percent confidence interval [32 to 47 percent]) respectively. Median overall survival was 14 months longer in the adjuvant chemotherapy group compared to the control group of patients with ERCC1-negative tumors (56 and 42 months respectively, FIG. 2B). Disease-free survival in patients with ERCC1-negative tumors was also significantly higher in the chemotherapy group compared to patients randomized to observation (adjusted hazard ratio for recurrence or death, 0.69; 95 percent confidence interval [0.53 to 0.90], P<0.007) (FIG. 2C).

There was no survival difference between the adjuvant chemotherapy group and the control group among patients with ERCC1-positive tumors (adjusted hazard ratio for death, 1.18; 95 percent confidence interval [0.87 to 1.61], P=0.29) (Table 3, FIG. 2D).

When the analysis focused exclusively on patients in the control group, the 5-year overall survival rate was significantly higher in patients with ERCC1-positive tumors (46 percent, 95 percent confidence interval [37 to 55 percent]) than in patients with ERCC1-negative tumors (39 percent, 95 percent confidence interval [32 to 47 percent]), with an adjusted hazard ratio of 0.65, 95 percent confidence interval [0.48 to 0.89], P<0.008 (Table 3).

### Example 7

Alternative roles for ERCC1 beyond NER are still currently emerging. It is now well established that ERCC1 is an important factor for DNA interstrand cross-link repair (ICL-R) (Usanova et al 2010), as well as for DNA double-strand breaks (DSB) repair via HR (homologous recombination) subpathway SSA (single-strand annealing) (Motycka, 2004), also via NHEJ (non-homologous end-joining) subpathway MMEJ (microhomology-mediated end-joining) (Ahmad 2008; De Silva I. U. et al, 2002; Sargent et al, 2000) and via activation of the FA (Fanconi anemia) pathway by permitting FANCD2 focus formation (McCabe 2008, Naim 2013). ERCC1/XPF also acts to limit non-LTR retrotransposition (Gasior 2008).

To achieve all these functions the ERCC1/XPF complex interacts with a wide range of partners. ERCC1 is catalytically inactive but indispensable for the activity of the complex and regulates DNA-/protein-protein interactions, whereas XPF provides the endonuclease activity and is involved in DNA binding and additional protein-protein interactions (see McNeil and Melton 2012 for review). ERCC1 interacts directly with XPA (xeroderma pigmentosum group A) (Li, 1994) and MAD2A (Mitotic arrest deficient 2) (Fung, 2008) for NER, MSH2 (MutS protein homolog 2) (Lan, 2004) and FANCG (Fanconi anemia complementation group G) for ICL-R (Wang and Lambert, 2010). XPF binds to RPA (replication protein A) for NER (Bessho, 1997; Fisher, 2011), TRF2 (telomeric repeat-binding factor 2) for telomere maintenance (Zhu, 2003; Wu 2008), SLX4/BTBD12 (BTB domain-containing protein 12) for ICL-R (Svendsen 2009; Munoz 2009) and RAD52 for SSA (Motycka, 2004). Importantly, the pharmacological inhibition of the ERCC1/XPF interaction leads to increased therapeutic effect from alkylating agents such as cisplatine in cancer cells (Jordheim, 2013).

A non-repair related role for ERCC1 was also proposed in mitosis process. Studies reported that cells from ERCC1-deficient mice harboured increased genome instability, chromosome aberrations, multinucleation, enlarged nuclei with various degrees of ploidy, disruptions in cell cycle, a decrease rate of cell proliferation, and cytoplasmic morphologic modifications (Weeda, 1997; Melton, 1998; Chipchase, 2002). Recently, ERCC1 knockdown in human cells confirmed these observations independently of XPF (Rageul, 2011) or linked to XPF and kinesin Eg5 binding (Li Jing Tan 2012). Although, it is unclear if the ERCC1 impact on mitosis process is dependent or not on ERCC1 DNA-repair functions since unrepaired endogenous DNA damage could lead to these types of abnormal cellular morphology.

ERCC1 knockout cells have been widely studied from mice and CHO (Chinese hamster ovary) cells and gave important knowledge about ERCC1 functions and alternative roles beyond NER but a human ERCC1 knockout cell line had never been reported. Using Zinc-finger targeting nucleases, our group established the first model of human cancer cells ERCC1-deficient. We recently published the establishment of these A549 (lung carcinoma human cell line) ERCC1-deficient cells that displayed a high sensitivity to cisplatin accompanied with a low rate of cisplatin DNA-adduct repair by NER (Friboulet NEJM 2013). We identified that only the reintroduction of the ERCC1-202 isoform rescued NER activity and capacity to counteract cisplatin treatment (Friboulet et al, NEJM2013). These data provided important insight into the relative function of the four ERCC1 isoforms for removal of cisplatin DNA-adducts and the way they might influence patient survival.

Since the four isoforms are expressed in human samples, we tempted here to elucidate the implication of these different ERCC1-isoforms on ERCC1 functions beyond NER and DNA repair. We searched for negative dominant isoform, we analysed the interactions between ERCC1 isoforms and previously identified ERCC1-interacting partners, we examined their cellular localization and finally we investigated the influence of each isoform on the cellular mitotic process.

### Materials and Methods

RNA Extraction and Quantitative Reverse Transcriptase PCR (qRT-PCR)

For ERCC1 isoform mRNA analysis we used frozen patient samples from the CHEMORES initiative (Chemotherapy resistance consortium) previously published (Friboulet, 2011).

The RNA extraction was performed with Qiagen RNeasy Mini Kit (74004; Qiagen). Total RNA (1 μg) was reverse-transcribed using the MuIV reverse transcriptase (Applied Biosystems). We designed specific TaqMan primers and probes for the different ERCC1 transcripts (sequences previously published) (Friboulet NEJM 2013). The relative expression of ERCC1 isoform mRNA was determined using the Ct value and the 2-ΔΔCt method. The data were presented as the fold-change in gene expression normalized to total ERCC1 mRNA.

### Cell Lines and Proliferation Assays

Cells were grown in DMEM medium (Gibco-Invitrogen) supplemented with 10% fetal calf serum (FCS). Two different tests were used to assess cell viability:

The clonal growth of NSCLC cells was assessed by plating 500 and 1000 cells per well in six-well plates treated with low concentrations of cisplatin (50 to 2000 nM) for 2 to 3 weeks. Cell colonies were stained with a solution of crystal violet in methanol. Dried plates were then scanned and digitized to allow optical magnification and precise quantification of well area stained.

Alternatively, the cell proliferation was determined in a short-term assay based on the reduction of WST-1 (water-soluble tetrazolium salt) (Roche Molecular), after 48 hours of treatment with various concentrations of cisplatin (from 0.2 to 40 μM) and mitomycin-C (from 0.75 to 100 nM) and the IC50 was determined.

### Cell Cycle and DNA Content

To study effect of cisplatin on cell cycle arrest, cells were treated with 30 nM or 300 nM of cisplatin for 48 h. For high DNA content analysis, cells were blocked in G2/M cell cycle phase with Karyomax colcemid solution (Gibco-Invitrogen) at 0.1 μg/ml for 6 h.

DNA content was determined in ethanol-fixed cells, stained with propidium iodide and analyzed using a Becton Dickinson FACScalibur flow cytometer and the CellQuest Pro software.

### Cell Protein Extraction and Western Blot Analysis

Proteins were extracted by lysis in RIPA buffer (50 mM Tris, 150 mM NaCl, 5 mM EDTA, 0.5% sodium deoxycholic acid, 0.5% NP-40, 0.1% SDS) supplemented with a protease inhibitor cocktail (Complete; Roche Molecular). For nucleus and cytoplasm protein, a first extraction and separation was done with a buffer containing 10 mM HEPES, 10 mM KCl, 1 mM DTT, 1 mM PMSF and protease inhibitor cocktail supplemented with 0.3% NP40. The nucleus fraction was next resuspended in a buffer containing 20 mM HEPES and 400 mM NaCl. Protein were then separated by SDS-PAGE and transferred to nitrocellulose membranes by the iBlot® 7-Minute Blotting System (Invitrogen). Blots were incubated with primary and secondary peroxidase-conjugated antibodies and chemiluminescent detection was done using the Dura HRP Substrate (Thermo scientific).

The antibodies used were ERCC1-3H11 (sc53281; Santa Cruz), XPF-3F2, TRF2, FANCG, Lamin-B1, Eg5, MAD2A (ab85140, ab13579, ab54645, ab16048, ab51976, ab10691; abcam), ERCC1-8F1 (MS-671P1; MM France), SLX4 (H00084464; abnova), XPA (MA1-21460; pierce), MSH2 (orb16010; BIORBYT), MMS19 (66049; proteintech) and β-actin or β-tubulin antibodies (A5441, T8328; Sigma-Aldrich) for loading controls.

### Diff Quik Stain

For cells morphology study, cells were fixed and stained with Diff Quik kit (130832; DadeBehring/Siemens) according to the manufacturer's instructions.

Treatments of Cells with Pharmacological Reagents

For proteasome inhibition, cells were treated with MG132 (Merck) at 2 μM for 24 h.

For video microscopy, cells were first stained with cell tracker green 2.5 μM for 30 min (C2925; Invitrogen) and then stained with Hoechst 1/8000 (62249SPCL; thermo scientific).

### α and γ Tubulin Immunofluorescence Staining

Microtubules were first stabilized in PHEM buffer and then cells were fixed and permeabilized in cold methanol for 5 min. After washing with PBS 0.1% Tween, and with IFF buffer (PBS, BSA 2%, FCS 5%), cells were incubated with primary antibody [1:200 for β-tubulin antibody (T8328; Sigma) and 1:1000 for γ-tubulin (ab27076; abcam)] in IFF for 45 min at room temperature. Cells were washed with PBS 0.1% Tween and incubated with secondary fluorescent antibody Alexa fluor (Invitrogen) in IFF for 30 min at room temperature. After washing with PBS 0.1% Tween, slides were mounted with Antifade ProLong with DAPI (Invitrogen).

### ERCC1-XPF Immunofluorescence Staining

Cells were fixed and permeabilized in formol and SDS 0.1% and then washed with PBS. After blocking with BSA 5%, cells were incubated with primary antibodies (1:200) ERCC1-FL297 (sc-10785; Santa Cruz), XPF-3F2 (ab85140; Abcam) in blocking solution for 1 h at 37° C. Cells were washed with PBS and incubated with secondary fluorescent antibody Alexa fluor (Invitrogen) in blocking solution for 1 h at 37° C. After washing with PBS, slides were mounted with Antifade ProLong with DAPI (Invitrogen).

### Proximity Ligation Assay (PLA)

Protein interactions were studied using the Duolink II proximity ligation assays (PLA) kit (Olink, Uppsala, Sweden). Coverslips were processed according to the manufacturer's instructions. In brief, the cells were fixed with methanol, permeabilized with triton, stained with the primary antibodies, and then incubated with the secondary oligonucleotide-linked antibodies. The oligonucleotides were hybridized, ligated, amplified, and detected using a fluorescent probe.

For all IF staining images were acquired an Inverted Ti-E fluorescence microscope (Nikon) and were processed with ImageJ software.

### Results

### Absence of Negative Dominant Isoform for Cisplatin Sensitivity.

We previously determined that only ERCC1-202 isoform appeared able to allow removal of cisplatin-DNA adducts and to improve survival after cisplatin treatment (Friboulet 2013). Since ERCC1-203 isoform had been proposed to be a negative dominant of ERCC1 DNA repair function, we tried to elucidate what influence could have ERCC1-201, 203 and 204 isoforms on ERCC1-202 DNA repair capacity.

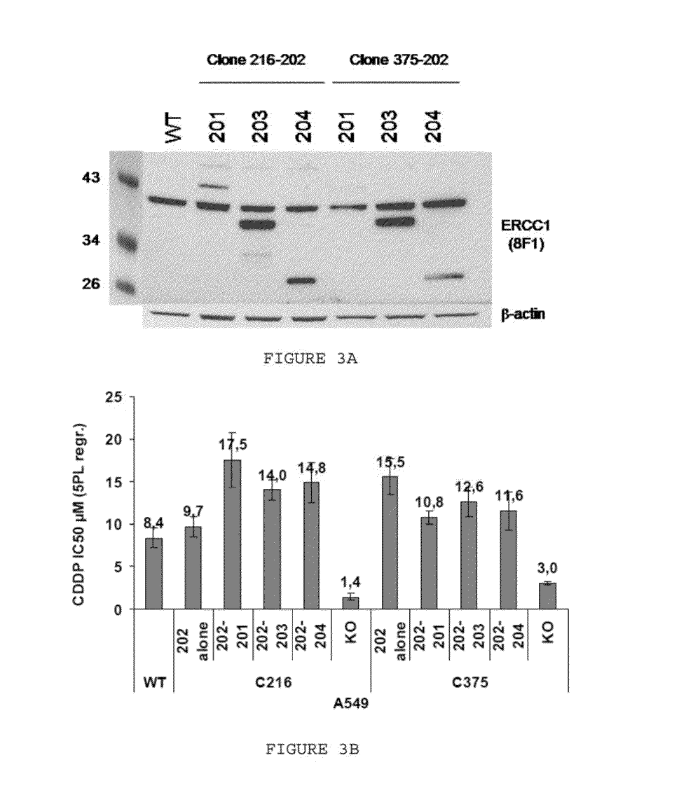

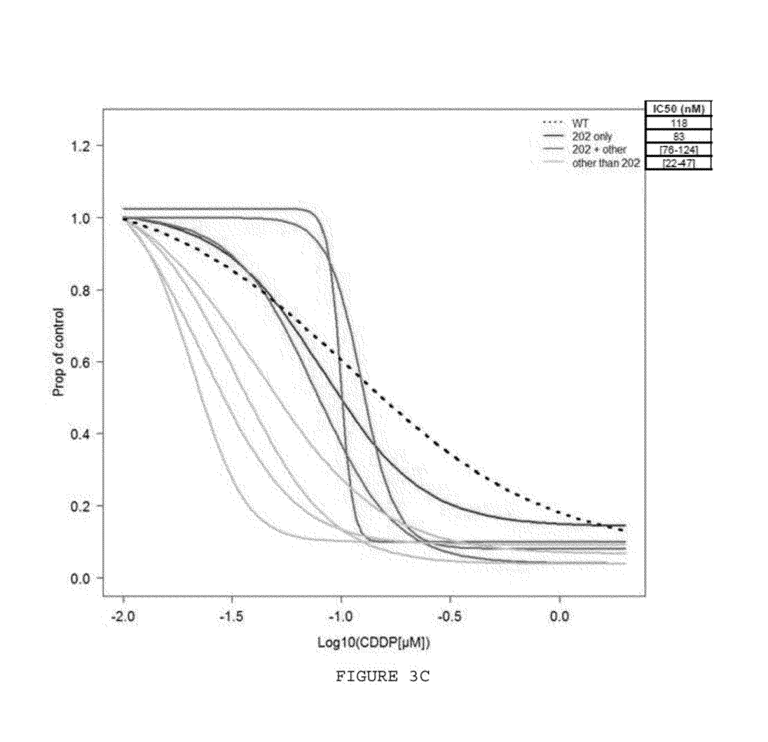

We selectively re-expressed each isoform with the ERCC1-202 isoform (FIG. 3A). Cell viability analysis after cisplatin exposure in these cells did not bring out any suppressive effect of other isoforms. Indeed, none of the other isoforms decreased cisplatin resistance (IC50) conferred by isoform 202 (FIG. 3B). These data were confirmed by clonogenic growth experiments (FIG. 3C).

### Cellular Localization of ERCC1 Isoforms.

It has been shown in XPF mutant cell lines, that ERCC1-XPF was detected in the cytoplasm of cells likely due to protein misfolding (Ahmad, 2010). We thus explored the cellular localization and the protein stability of the different ERCC1 isoforms.

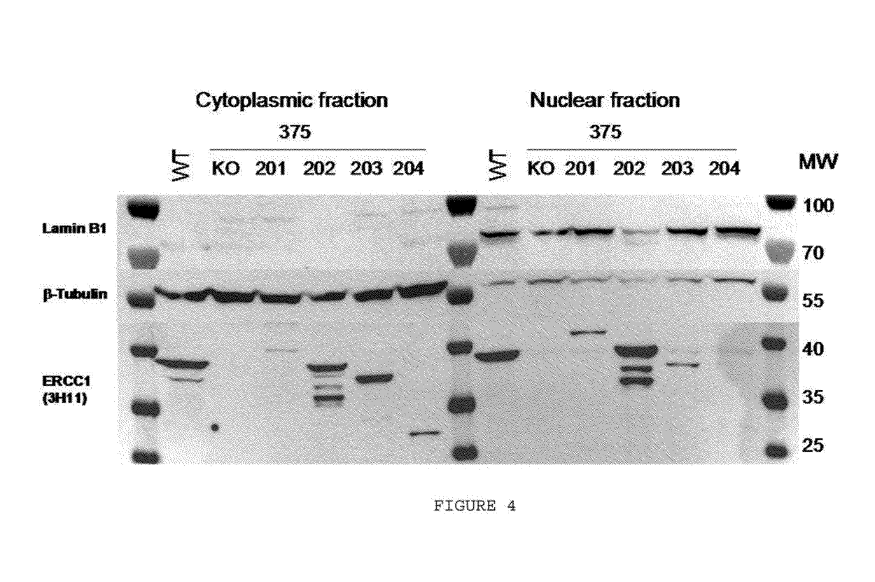

Immunofluorescence detection of ERCC1 protein isoforms suggested a main nuclear localization of ERCC1-201 and -202 isoforms whereas ERCC1-203 and -204 isoform were also detected in the cytoplasm. ERCC1 protein isoforms detection by western blot after cellular fractionation confirmed their differential cellular localization (FIG. 4).

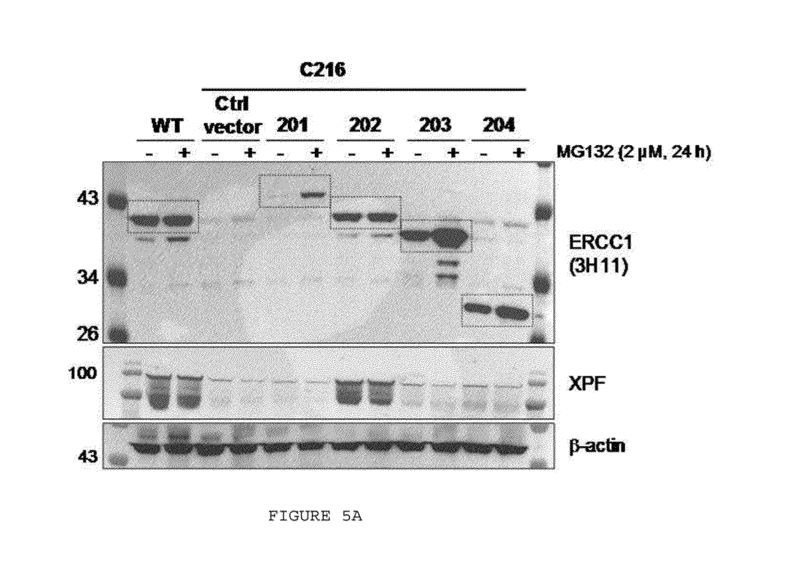

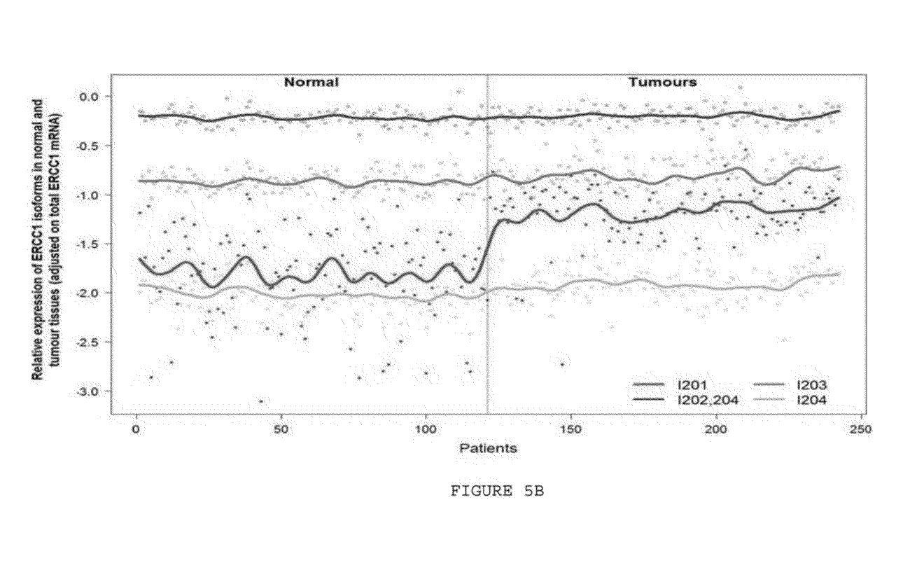

We thus explored the stability of each protein isoform using proteasome inhibition. This inhibition leaded to an increase expression level of ERCC1-201, -203 and -204 suggesting these isoforms are unstable and quickly degraded probably due to protein misfolding (FIG. 5A). These results could suggest the uselessness of these isoforms for human cells.

ERCC1-201 mRNA Isoform is Upregulated in Tumours Samples.

We previously detected ERCC1 isoforms at the mRNA level in 123 NSCLC patients belonging to the Chemores consortium (Friboulet, 2011). To investigate a possible role of ERCC1 isoforms in the oncogenic process we compared the expression of ERCC1 isoforms between matched tumour and normal specimens by qRT-PCR. The four isoforms were detected at the mRNA level, both in tumor and normal tissues (FIG. 5B). Interestingly, a significant increase in ERCC1-201 isoform expression was observed in all tumor tissues compared to normal counterparts. Other isoforms were homogenously expressed in normal and tumor tissues. This overexpression of ERCC1-201 isoform in tumour samples could suggest an oncogenic role of this isoform.

### ERCC1-202 Isoform is Essential for Proper Chromosome Segregation.

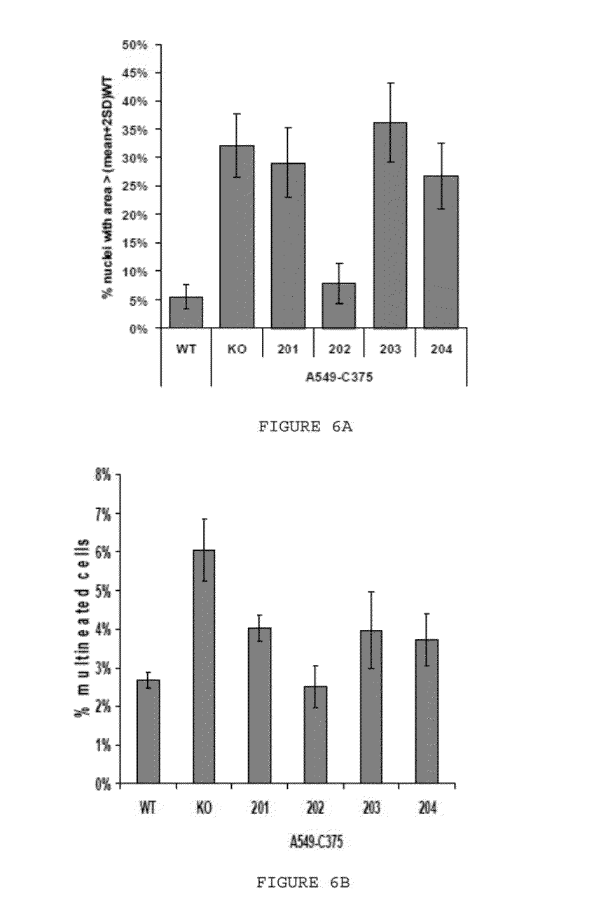

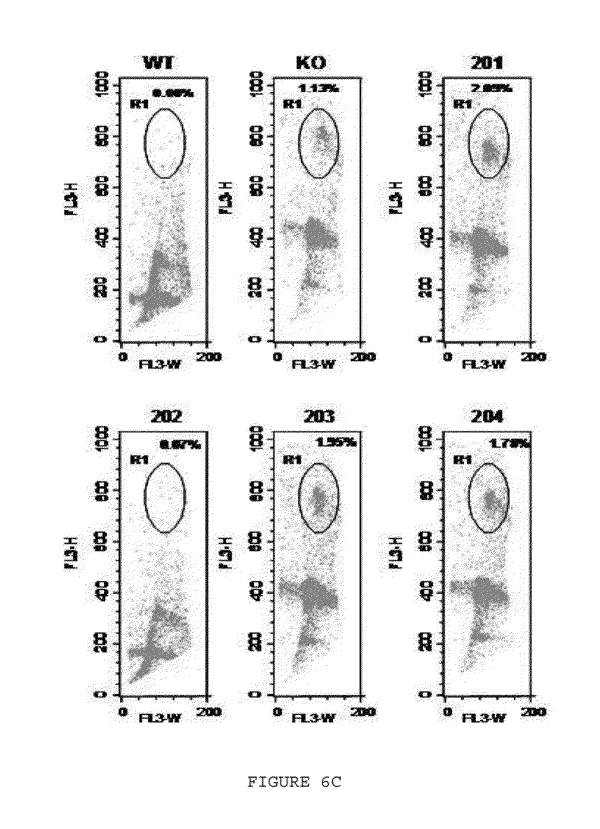

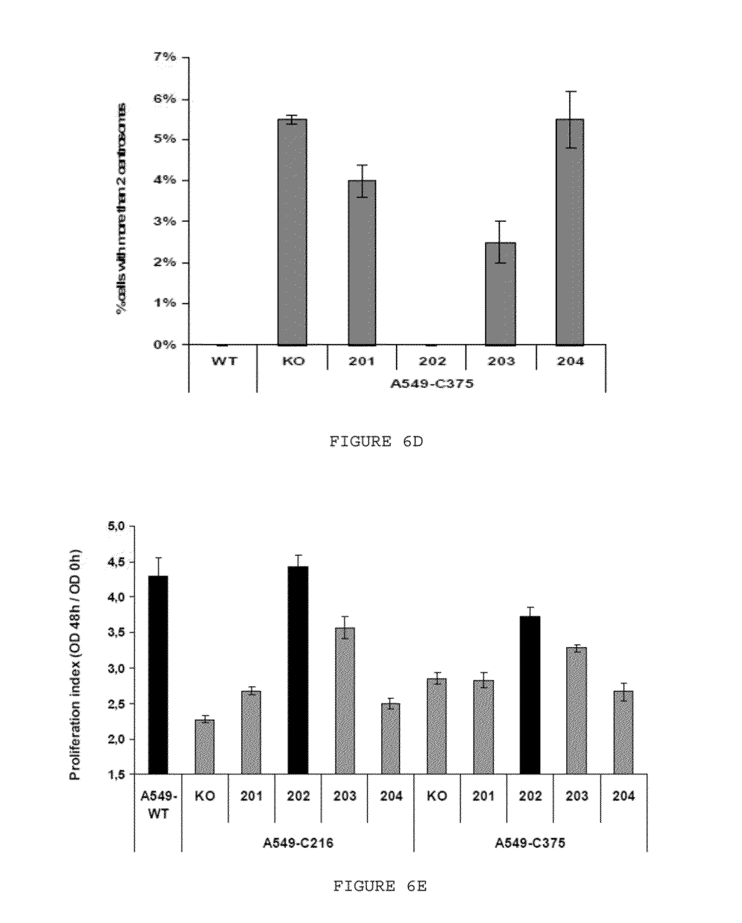

Studies reported that ERCC1-deficient mouse cells and human cells after ERCC1 knockdown harboured nuclear and cytoplasm morphologic alterations at least in part due to abnormal mitosis. We indeed observed strong morphologic modifications in ERCC1 deficient cells: bulky cells with huge nucleus, multinucleation and important spreading of the cytoplasm (not shown). We observed these morphologic modifications in cells re-expressing isoforms 201, 203 and 204. Only ERCC1-202 isoform prevented the appearance of cells with giant nucleus and multinucleated cells (FIGS. 6A and 6B).

Accordingly, by flow cytometry in cells blocked in metaphase by Colcemid microtubule-depolymerizing drug, we observed a significant increase in the percentage of polyploidy cells (more than 4N DNA) in the absence of ERCC1-202 isoform expression (FIG. 6C). After cisplatin treatment (30 and 300 nM for 48 hours) cell lines without ERCC1-202 isoform expression remained largely (60-80%) blocked in G2/M cell cycle phase (not shown). Altogether, these data confirmed that the ERCC1 functional-deficiency may induce aneuploidy.

Improper chromosomes alignment for metaphase was proposed to explain multinucleation occurrences in ERCC1 attenuated cells. We analysed mitotic spindle shape in proliferating cells by alpha- and gamma-tubulin immunofluorescent staining. We observed abnormal centrosomes number and many DNA bridges in cells without ERCC1-202 isoform expression (FIG. 6D). DNA bridges have been shown to occlude the division site and are a common cause for cytokinesis failure. It is therefore possible that the increase in DNA bridges observed in ERCC1-deficient cells leads to a failure in cell division. By monitoring the cell division in time-laps experiments, we indeed observed impaired cytokinesis leading to daughter cells fusion (not shown). Accordingly, these mitosis defects reduced strongly the proliferation rate in cells without ERCC1-202 isoform expression (FIG. 6E). Our results clearly suggested that only ERCC1-202 isoform restored chromosome segregation accuracy.

### ERCC1 Isoforms Function in ICL-R and HR.

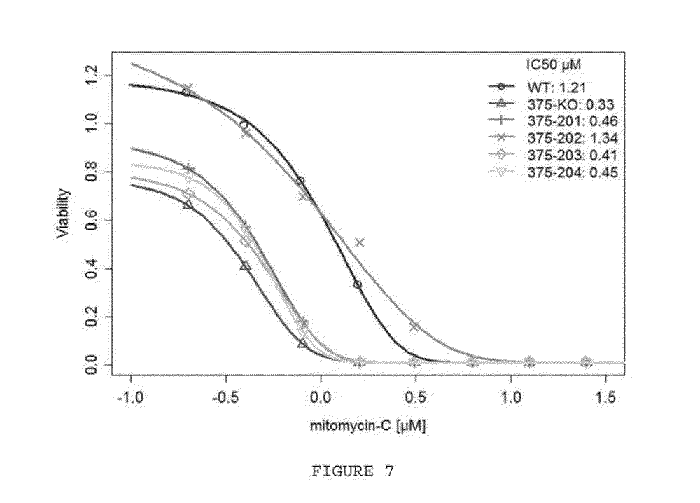

It is clearly established that Fanconi anemia (FA) pathway-deficient cells are hypersensitive to DNA crosslinking agent such as mitomycin C (MMC). More recently, it has been shown that disruption of the FA pathway results in cytokinesis failure with frequent DNA bridges and an increase in multinucleated cells (Vinciguerra, 2010). It can be speculated that cytokinesis failure observed in ERCC1-deficient cells could arise from defect in ICL-R. We therefore investigated the ICL-R ability of the different ERCC1 isoforms by determining the mitomycin-C IC50-values of cells expressing unique isoform. As we previously observed for cisplatin treatment, MMC cell resistance was rescued only by ERCC1-202 isoform re-expression in short term (48 h) proliferation assays (FIG. 7). It is therefore possible that unrepaired ICL damage in ERCC1-202 deficient cells lead to DNA bridges and cytokinesis failure.

By immunofluorescence we analysed the amount of H2AX and Rad51 foci after mitomycin-C treatment. Accordingly, we observed an increase in ERCC1-202 isoform expression (not shown).

### Interacting Abilities of ERCC1 Isoforms.

Studies suggested that ERCC1 and XPF are unstable in the absence of each partner in mammalian cells (Arora, 2010). Indeed in our ERCC1-deficient cells the expression level of XPF was highly reduced (FIG. 5A). We noticed that only ERCC1-202 isoform expression rescued XPF protein expression levels. Considering many works that proposed ERCC1/XPF as a necessary complex to ensure stability of both proteins we speculated that only isoform 202 was able to interact with and protect against XPF degradation.

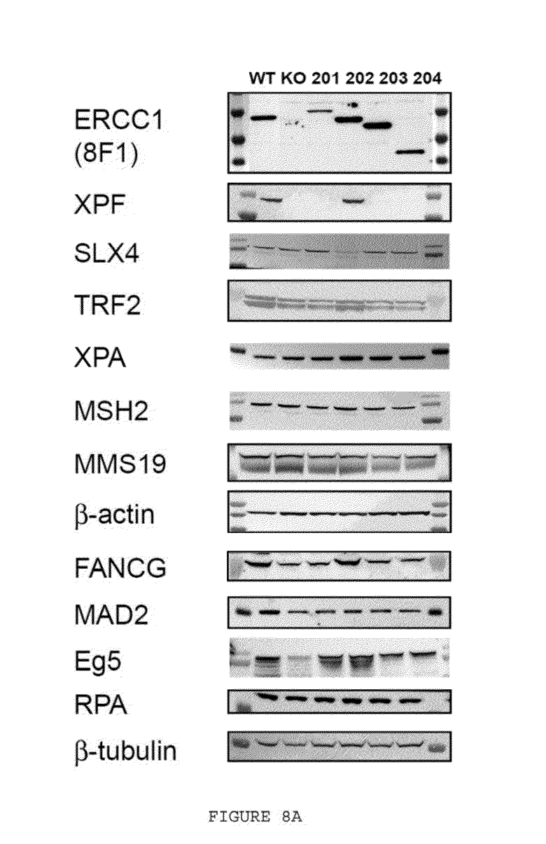

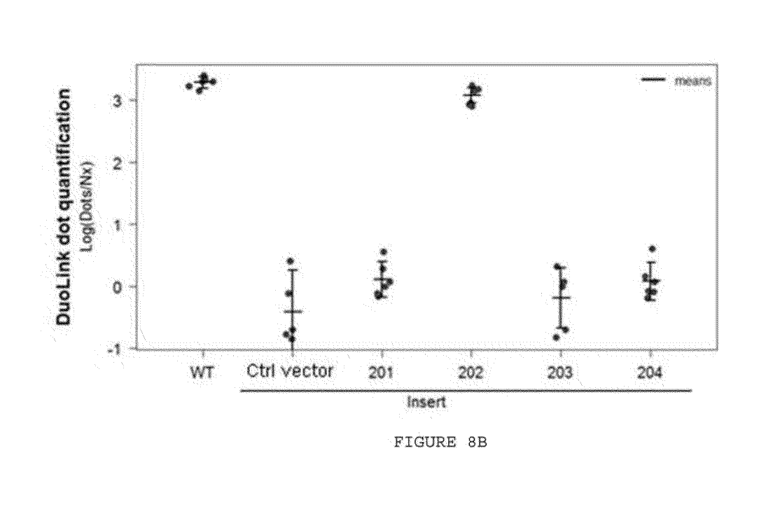

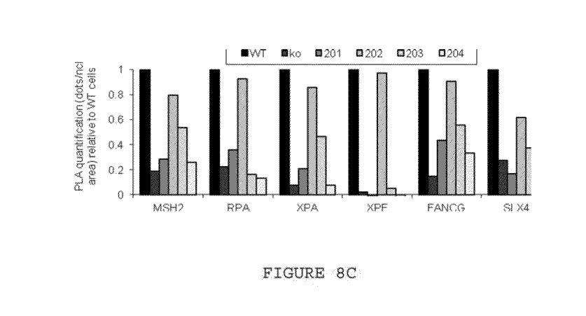

The expression level of others previously described ERCC1 interacting proteins was analyzed in cells expressing only one ERCC1 isoform. Loss of ERCC1 expression and isoform expression rescue did not modify the protein expression level of XPA, SLX4, TRF2, FANCG, MAD2A, Eg5 or MSH2 proteins (FIG. 8A).

Using proximity ligation assays (PLA-Duolink) technology, we investigated the binding ability of ERCC1 isoforms with ERCC1 interacting proteins. High number of ERCC1/XPF heterodimers were detected only in cells expressing ERCC1-202 isoform (FIG. 8B). These data provided evidence that XPF protein is unstable in the absence of ERCC1-202 isoform and that only this isoform could form a stable heterodimer complex with XPF.

Similarly, we identified ERCC1/XPA, ERCC1/MSH2, ERCC1/FANCG, ERCC1/SLX4, ERCC1/Eg5, ERCC1/MAD2A and ERCC1/TRF2 complexes only with ERCC1-202 isoform (FIG. 8C). All together these data suggested that ERCC1-protein complexes required ERCC1-202 isoform expression.

### Discussion

Despite the huge interest of ERCC1 biomarker in the cancer research community, the DNA repair functionality and alternative roles of the different human ERCC1 isoforms remained largely uncharacterized. In order to study ERCC1 isoforms individually we established the first ERCC1 knockout NSCLC cell lines (Friboulet NEJM 2013).

For the first time we brought out that several previously identified functions of ERCC1 are realized by the same ERCC1 isoform, the ERCC1-202. We have shown that ERCC1 201, 203 and 204 isoforms were unable to achieved ERCC1 functions and interactions and none of them seemed to be a negative dominant of the ERCC1-202 isoform for cisplatin DNA damage repair. The reason for a difference from previous studies proposing a negative role of ERCC1-203 isoform is not known but could be due to a difference in experimental methods used and the fact that completely abolished ERCC1 basal expression appeared essential in our hands to elucidate the biological influence of each isoform.

XPF is essential for the nuclease activity of the ERCC1/XPF complex. Since only ERCC1-202 isoform formed heterodimer with XPF, all functions of the complex linked to nuclease activity can only be observed in cells expressing ERCC1-202 isoform. Other isoforms could be implicated in non-nuclease linked activities but further work is needed to elucidate the specific biological function of each of the other ERCC1 isoforms that seemed to be widely expressed in human samples. The role of specifically overexpression of 201 isoform mRNA in tumors also remains to be clarified.

Cells deficient in ERCC1 protein displayed high rates of multinucleated cells as a result of DNA bridges and cytokinesis failure. It has been speculated that unrepaired DNA damages may be the source of elevated chromatin bridges and cytokinesis failure. We can therefore hypothesize that ERCC1 implication in mitosis could at least in part account for the nuclease activity of the ERCC1/XPF complex in DNA repair.

Our data clearly suggested that the development of a diagnostic method recognizing ERCC1/XPF heterodimers should match to functional ERCC1-202 isoform quantification only.

We claim:

1. An in vitro method for detecting the susceptibility of a tumor cell to a chemotherapy, said method comprising the step of measuring the expression level of the isoform 202 of the ERCC1 protein.

2. The method according to claim 1, wherein the expression level of the isoform 202 of the ERCC1 protein is measured by assessing the mRNA level of the isoform 202 of the ERCC1 protein.

3. The method according to claim 2, wherein said mRNA level is measured by RT-PCR or in situ hybridization.

4. The method according to claim 2, wherein said mRNA is SEQ ID NO:1.

5. The method according to claim 1, wherein the expression level of the isoform 202 of the ERCC1 protein is measured by means of an immunohistochemistry assay or of an immunofluorescence assay.

6. The method according to claim 1, wherein the expression level of the isoform 202 of the ERCC1 protein is measured by means of an immunohistochemistry assay performed on a formalin-fixed paraffin-embedded tumor sample.

7. The method according to claim 6, wherein said immunohistochemistry assay uses a monoclonal ERCC1 antibody recognizing specifically the isoform 202 of the ERCC1 protein or an antibody recognizing specifically an heterodimer selected from the group consisting of: ERCC1/XPF, ERCC1/XPA, ERCC1/MSH2, ERCC1/FANCG, ERCC1/SLX4, ERCC1/Eg5, ERCC1/MAD2A, and ERCC1/TRF2.

8. The method according to claim 6, further including the steps of:
(a) obtaining slides from formalin-fixed paraffin-embedded tumor samples;
(b) retrieving epitope in buffer;
(c) incubating slides with a monoclonal ERCC1 antibody recognizing specifically the isoform 202 of the ERCC1 protein; or with an antibody recognizing an heterodimer selected from the group consisting of: ERCC1/XPF, ERCC1/XPA, ERCC1/MSH2, ERCC1/FANCG, ERCC1/SLX4, ERCC1/Eg5, ERCC1/MAD2A, and ERCC1/TRF2; or with an antibody recognizing specifically the XPF protein, the XPA protein, the MSH2 protein (isoforms 1 or 2), the FANCG protein, the SLX4 protein, the Eg5 protein, the MAD2A protein, or the TRF2 protein;
(d) determining an amount of binding antibodies on the formalin-fixed paraffin-embedded tumor samples, using the amount of binding antibodies on an internal positive control as a reference;
(e) determining a percentage of labeled nuclei on the formalin-fixed paraffin-embedded tumor samples;
(f) multiplying the value estimated in step (d) with the value estimated in step (e); and
(g) determining a platinum-based chemotherapy regimen by comparing the value obtained in step (f) to a median score of the values obtained in step (f).

9. The method according to claim 8, wherein the internal positive control consists of stroma cells surrounding the tumor area.

10. The method according to claim 1, wherein the expression level of the isoform 202 of the ERCC1 protein is measured by means of an immunofluorescence assay performed on individual tumor cells.

11. The method according to claim 1, wherein said expression level is measured by means of an antibody that recognizes specifically the isoform 202 of the ERCC1 protein; or with an antibody recognizing specifically an heterodimer selected from the group consisting of: ERCC1/XPF, ERCC1/XPA, ERCC1/MSH2, ERCC1/FANCG, ERCC1/SLX4, ERCC1/Eg5, ERCC1/MAD2A, and ERCC1/TRF2; or with an antibody recognizing specifically the XPF protein, the XPA protein, the MSH2 protein (isoforms 1 or 2), the FANCG protein, the SLX4 protein, the Eg5 protein, the MAD2A protein, or the TRF2 protein.

12. The method according to claim 1, wherein said tumor is a non-small-cell lung cancer.

13. The method according to claim 1, wherein said chemotherapy is a platinum-based cancer chemotherapy.

14. The method according to claim 1, wherein said chemotherapy is based on cisplatin.

15. The method according to claim 1, wherein said chemotherapy is cisplatin associated with other chemotherapeutic agents.

16. The method according to claim 1, wherein said chemotherapy is cisplatin with etoposide or a vinca alkaloid.

17. The method according to claim 1, wherein said patient had undergone a surgical resection of its tumor.

18. A kit for the detection or quantification of the isoform 202 of the ERCC1 protein, comprising:
(a) an antibody that recognizes specifically the isoform 202 of the ERCC1 protein, or
(b) an antibody recognizing specifically an heterodimer selected from the group consisting of: ERCC1/XPF, ERCC1/XPA, ERCC1/MSH2, ERCC1/FANCG, ERCC1/SLX4, ERCC1/Eg5, ERCC1/MAD2A, and ERCC1/TRF2; or
(c) an antibody recognizing specifically the XPF protein, the XPA protein, the MSH2 protein (isoforms 1 or 2), the FANCG protein, the SLX4 protein, the Eg5 protein, the MAD2A protein, or the TRF2 protein.

19. An in vitro method for detecting the susceptibility of a tumor cell to a chemotherapy, using the kit of claim 18.


SEQUENCE LISTING (17 sequences)

SEQ ID NO: 1 - DNA, 1101 bp, homo sapiens
  misc_feature: mRNA sequence of the ERCC1 isoform 202 (corresponding to NM_001983.3)
ccggaagtgctgcgagccctgggccacgctggccgtgctggcagtgggccgcctcgatccctctgcagtctttcccttgaggctccaagaccagcaggtgaggcctcgcggcgctgaaaccgtgaggcccggaccacaggctccagatggaccctgggaaggacaaagagggggtgccccagccctcagggccgccagcaaggaagaaatttgtgatacccctcgacgaggatgaggtccctcctggagtggccaagcccttattccgatctacacagagccttcccactgtggacacctcggcccaggcggcccctcagacctacgccgaatatgccatctcacagcctctggaaggggctggggccacgtgccccacagggtcagagcccctggcaggagagacgcccaaccaggccctgaaacccggggcaaaatccaacagcatcattgtgagccctcggcagaggggcaatcccgtactgaagttcgtgcgcaatgtgccctgggaatttggcgacgtaattcccgactatgtgctgggccagagcacctgtgccctgttcctcagcctccgctaccacaacctgcacccagactacatccatgggcggctgcagagcctggggaagaacttcgccttgcgggtcctgcttgtccaggtggatgtgaaagatccccagcaggccctcaaggagctggctaagatgtgtatcctggccgactgcacattgatcctcgcctggagccccgaggaagctgggcggtacctggagacctacaaggcctatgagcagaaaccagcggacctcctgatggagaagctagagcaggacttcgtctcccgggtgactgaatgtctgaccaccgtgaagtcagtcaacaaaacggacagtcagaccctcctgaccacatttggatctctggaacagctcatcgccgcatcaagagaagatctggccttatgcccaggcctgggccctcagaaagcccggaggctgtttgatgtcctgcacgagcccttcttgaaagtaccctgatgaccccagctgccaaggaaacccccagtgtaataataaatcgtcctcccaggccaggctc

SEQ ID NO: 2 - protein, 297 aa, homo sapiens
  MISC_FEATURE: amino acid sequence of the ERCC1 isoform 202 (corresponding to NP_001974.1)
MDPGKDKEGVPQPSGPPARKKFVIPLDEDEVPPGVAKPLFRSTQSLPTVDTSAQAAPQTYAEYAISQPLEGAGATCPTGSEPLAGETPNQALKPGAKSNSIIVSPRQRGNPVLKFVRNVPWEFGDVIPDYVLGQSTCALFLSLRYHNLHPDYIHGRLQSLGKNFALRVLLVQVDVKDPQQALKELAKMCILADCTLILAWSPEEAGRYLETYKAYEQKPADLLMEKLEQDFVSRVTECLTTVKSVNKTDSQTLLTTFGSLEQLIAASREDLALCPGLGPQKARRLFDVLHEPFLKVP

SEQ ID NO: 3 - DNA, 1255 bp, homo sapiens
  misc_feature: mRNA sequence of the ERCC1 isoform 201 (corresponding to NM_202001.2)
aaaggggcgtggcgttacagagcctctagcgctgggtgttggggacctgacgctatggagctctcggagttttgtgggggacggctgtgagtggggggttcctgctgcgggatgagaacgtagacgccagtggctcactcgctcctggcaccttccctttcaggctccagatggaccctgggaaggacaaagagggggtgccccagccctcagggccgccagcaaggaagaaatttgtgatacccctcgacgaggatgaggtccctcctggagtggccaagcccttattccgatctacacagagccttcccactgtggacacctcggcccaggcggcccctcagacctacgccgaatatgccatctcacagcctctggaaggggctggggccacgtgccccacagggtcagagcccctggcaggagagacgcccaaccaggccctgaaacccggggcaaaatccaacagcatcattgtgagccctcggcagaggggcaatcccgtactgaagttcgtgcgcaatgtgccctgggaatttggcgacgtaattcccgactatgtgctgggccagagcacctgtgccctgttcctcagcctccgctaccacaacctgcacccagactacatccatgggcggctgcagagcctggggaagaacttcgccttgcgggtcctgcttgtccaggtggatgtgaaagatccccagcaggccctcaaggagctggctaagatgtgtatcctggccgactgcacattgatcctcgcctggagccccgaggaagctgggcggtacctggagacctacaaggcctatgagcagaaaccagcggacctcctgatggagaagctagagcaggacttcgtctcccgggtgactgaatgtctgaccaccgtgaagtcagtcaacaaaacggacagtcagaccctcctgaccacatttggatctctggaacagctcatcgccgcatcaagagaagatctggccttatgcccaggcctgggccctcagaaagtaagagctctgggaaagaacccaaggagttgggggaaggagagagccccaaataaacacaacctgagaccccaaagttttaaggtgaaaaaagaaccaaagaccagacacagtggcttccgcctgtaatcccaacattttgggaggccaaggcgggaggactgcttgaggccagaagttggagaccagcctgggcaagtggacacctcatttttactaaaaataaaaaaaactagctgggc

SEQ ID NO: 4 - protein, 323 aa, homo sapiens
  MISC_FEATURE: amino acid sequence of the ERCC1 isoform 201 (corresponding to NP_973730.1)
MDPGKDKEGVPQPSGPPARKKFVIPLDEDEVPPGVAKPLFRSTQSLPTVDTSAQAAPQTYAEYAISQPLEGAGATCPTGSEPLAGETPNQALKPGAKSNSIIVSPRQRGNPVLKFVRNVPWEFGDVIPDYVLGQSTCALFLSLRYHNLHPDYIHGRLQSLGKNFALRVLLVQVDVKDPQQALKELAKMCILADCTLILAWSPEEAGRYLETYKAYEQKPADLLMEKLEQDFVSRVTECLTTVKSVNKTDSQTLLTTFGSLEQLIAASREDLALCPGLGPQKVRALGKNPRSWGKERAPNKHNLRPQSFKVKKEPKTRHSGFRL

SEQ ID NO: 5 - DNA, 960 bp, homo sapiens
  misc_feature: mRNA sequence of the ERCC1 isoform 203 (corresponding to NM_001166049.1)
tggcagtgggccgcctcgatccctctgcagtctttcccttgaggctccaagaccagcaggtgaggcctcgcggcgctgaaaccgtgaggcccggaccacaggctccagatggaccctgggaaggacaaagagggggtgccccagccctcagggccgccagcaaggaagaaatttgtgatacccctcgacgaggatgaggtccctcctggagtggccaagcccttattccgatctacacagagccttcccactgtggacacctcggcccaggcggcccctcagacctacgccgaatatgccatctcacagcctctggaaggggctggggccacgtgccccacagggtcagagcccctggcaggagagacgcccaaccaggccctgaaacccggggcaaaatccaacagcatcattgtgagccctcggcagaggggcaatcccgtactgaagttcgtgcgcaatgtgccctgggaatttggcgacgtaattcccgactatgtgctgggccagagcacctgtgccctgttcctcagcctccgctaccacaacctgcacccagactacatccatgggcggctgcagagcctggggaagaacttcgccttgcgggtcctgcttgtccaggtggatgtgaaagatccccagcaggccctcaaggagctggctaagatgtgtatcctggccgactgcacattgatcctcgcctggagccccgaggaagctgggcggtacctggagacctacaaggcctatgagcagaaaccagcggacctcctgatggagaagctagagcaggacttcgtctcccggtctctggaacagctcatcgccgcatcaagagaagatctggccttatgcccaggcctgggccctcagaaagcccggaggctgtttgatgtcctgcacgagcccttcttgaaagtaccctgatgaccccagctgccaaggaaacccccagtg

SEQ ID NO: 6 - protein, 273 aa, homo sapiens
  MISC_FEATURE: amino acid sequence of the ERCC1 isoform 203 (corresponding to NP_001159521.1)
MDPGKDKEGVPQPSGPPARKKFVIPLDEDEVPPGVAKPLFRSTQSLPTVDTSAQAAPQTYAEYAISQPLEGAGATCPTGSEPLAGETPNQALKPGAKSNSIIVSPRQRGNPVLKFVRNVPWEFGDVIPDYVLGQSTCALFLSLRYHNLHPDYIHGRLQSLGKNFALRVLLVQVDVKDPQQALKELAKMCILADCTLILAWSPEEAGRYLETYKAYEQKPADLLMEKLEQDFVSRSLEQLIAASREDLALCPGLGPQKARRLFDVLHEPFLKVP

SEQ ID NO: 7 - DNA, 816 bp, homo sapiens
  misc_feature: mRNA sequence of the ERCC1 isoform 204 (corresponding to ENST00000423698.2)
tggcagtgggccgcctcgatccctctgcagtctttcccttgaggctccaagaccagcaggtgaggcctcgcggcgctgaaaccgtgaggcccggaccacaggctccagatggaccctgggaaggacaaagagggggtgccccagccctcagggccgccagcaaggaagaaatttgtgatacccctcgacgaggatgaggtccctcctggagtgaggggcaatcccgtactgaagttcgtgcgcaatgtgccctgggaatttggcgacgtaattcccgactatgtgctgggccagagcacctgtgccctgttcctcagcctccgctaccacaacctgcacccagactacatccatgggcggctgcagagcctggggaagaacttcgccttgcgggtcctgcttgtccaggtggatgtgaaagatccccagcaggccctcaaggagctggctaagatgtgtatcctggccgactgcacattgatcctcgcctggagccccgaggaagctgggcggtacctggagacctacaaggcctatgagcagaaaccagcggacctcctgatggagaagctagagcaggacttcgtctcccgggtgactgaatgtctgaccaccgtgaagtcagtcaacaaaacggacagtcagaccctcctgaccacatttggatctctggaacagctcatcgccgcatcaagagaagatctggccttatgcccaggcctgggccctcagaaagcccggaggctgtttgatgtcctgcacgagcccttcttgaaagtaccctgatgaccccagctgccaaggaaacccccagtg

SEQ ID NO: 8 - protein, 225 aa, homo sapiens
  MISC_FEATURE: amino acid sequence of the ERCC1 isoform 204 (corresponding to NSP00000394875.2)
MDPGKDKEGVPQPSGPPARKKFVIPLDEDEVPPGVRGNPVLKFVRNVPWEFGDVIPDYVLGQSTCALFLSLRYHNLHPDYIHGRLQSLGKNFALRVLLVQVDVKDPQQALKELAKMCILADCTLILAWSPEEAGRYLETYKAYEQKPADLLMEKLEQDFVSRVTECLTTVKSVNKTDSQTLLTTFGSLEQLIAASREDLALCPGLGPQKARRLFDVLHEPFLKVP

SEQ ID NO: 9 - protein, 916 aa, homo sapiens
  MISC_FEATURE: amino acid sequence of the XPF protein (corresponding to NP_005227.1). (Note: the official symbol of the corresponding coding gene is ERCC4).
MESGQPARRIAMAPLLEYERQLVLELLDTDGLVVCARGLGADRLLYHFLQLHCHPACLVLVLNTQPAEEEYFINQLKIEGVEHLPRRVTNEITSNSRYEVYTQGGVIFATSRILVVDFLTDRIPSDLITGILVYRAHRIIESCQEAFILRLFRQKNKRGFIKAFTDNAVAFDTGFCHVERVMRNLFVRKLYLWPRFHVAVNSFLEQHKPEVVEIHVSMTPTMLAIQTAILDILNACLKELKCHNPSLEVEDLSLENAIGKPFDKTIRHYLDPLWHQLGAKTKSLVQDLKILRTLLQYLSQYDCVTFLNLLESLRATEKAFGQNSGWLFLDSSTSMFINARARVYHLPDAKMSKKEKISEKMEIKEGEETKKELVLESNPKWEALTEVLKEIEAENKESEALGGPGQVLICASDDRTCSQLRDYITLGAEAFLLRLYRKTFEKDSKAEEVWMKFRKEDSSKRIRKSHKRPKDPQNKERASTKERTLKKKKRKLTLTQMVGKPEELEEEGDVEEGYRREISSSPESCPEEIKHEEFDVNLSSDAAFGILKEPLTIIHPLLGCSDPYALTRVLHEVEPRYVVLYDAELTFVRQLEIYRASRPGKPLRVYFLIYGGSTEEQRYLTALRKEKEAFEKLIREKASMVVPEEREGRDETNLDLVRGTASADVSTDTRKAGGQEQNGTQQSIVVDMREFRSELPSLIHRRGIDIEPVTLEVGDYILTPEMCVERKSISDLIGSLNNGRLYSQCISMSRYYKRPVLLIEFDPSKPFSLTSRGALFQEISSNDISSKLTLLTLHFPRLRILWCPSPHATAELFEELKQSKPQPDAATALAITADSETLPESEKYNPGPQDFLLKMPGVNAKNCRSLMHHVKNIAELAALSQDELTSILGNAANAKQLYDFIHTSFAEVVSKGKGKK

SEQ ID NO: 10 - protein, 273 aa, homo sapiens
  MISC_FEATURE: amino acid sequence of the XPA protein (corresponding to NP_000371.1)
MAAADGALPEAAALEQPAELPASVRASIERKRQRALMLRQARLAARPYSATAAAATGGMANVKAAPKIIDTGGGFILEEEEEEEQKIGKVVHQPGPVMEFDYVICEECGKEFMDSYLMNHFDLPTCDNCRDADDKHKLITKTEAKQEYLLKDCDLEKREPPLKFIVKKNPHHSQWGDMKLYLKLQIVKRSLEVWGSQEALEEAKEVRQENREKMKQKKFDKKVKELRRAVRSSVWKRETIVHQHEYGPEENLEDDMYRKTCTMCGHELTYEKM

SEQ ID NO: 11 - protein, 934 aa, homo sapiens [sequence omitted]

SEQ ID NO: 12 - protein, 868 aa, homo sapiens
  MISC_FEATURE: amino acid sequence of the MSH2 isoform 2 (corresponding to NP_001245210.1)
MGPAGAKNLQSVVLSKMNFESFVKDLLLVRQYRVEVYKNRAGNKASKENDWYLAYKASPGNLSQFEDILFGNNDMSASIGVVGVKMSAVDGQRQVGVGYVDSIQRKLGLCEFPDNDQFSNLEALLIQIGPKECVLPGGETAGDMGKLRQIIQRGGILITERKKADFSTKDIYQDLNRLLKGKKGEQMNSAVLPEMENQVAVSSLSAVIKFLELLSDDSNFGQFELTTFDFSQYMKLDIAAVRALNLFQGSVEDTTGSQSLAALLNKCKTPQGQRLVNQWIKQPLMDKNRIEERLNLVEAFVEDAELRQTLQEDLLRRFPDLNRLAKKFQRQAANLQDCYRLYQGINQLPNVIQALEKHEGKHQKLLLAVFVTPLTDLRSDFSKFQEMIETTLDMDQVENHEFLVKPSFDPNLSELREIMNDLEKKMQSTLISAARDLGLDPGKQIKLDSSAQFGYYFRVTCKEEKVLRNNKNFSTVDIQKNGVKFTNSKLTSLNEEYTKNKTEYEEAQDAIVKEIVNISSGYVEPMQTLNDVLAQLDAVVSFAHVSNGAPVPYVRPAILEKGQGRIILKASRHACVEVQDEIAFIPNDVYFEKDKQMFHIITGPNMGGKSTYIRQTGVIVLMAQIGCFVPCESAEVSIVDCILARVGAGDSQLKGVSTFMAEMLETASILRSATKDSLIIIDELGRGTSTYDGFGLAWAISEYIATKIGAFCMFATHFHELTALANQIPTVNNLHVTALTTEETLTMLYQVKKGVCDQSFGIHVAELANFPKHVIECAKQKALELEEFQYIGESQGYDIMEPAAKKCYLEREQGEKIIQEFLSKVKQMPFTEMSEENITIKLKQLKAEVIAKNNSFVNEIISRIKVTT

SEQ ID NO: 13 - protein, 622 aa, homo sapiens
  MISC_FEATURE: amino acid sequence of the FANCG protein (corresponding to NP_004620.1)
MSRQTTSVGSSCLDLWREKNDRLVRQAKVAQNSGLTLRRQQLAQDALEGLRGLLHSLQGLPAAVPVLPLELTVTCNFIILRASLAQGFTEDQAQDIQRSLERVLETQEQQGPRLEQGLRELWDSVLRASCLLPELLSALHRLVGLQAALWLSADRLGDLALLLETLNGSQSGASKDLLLLLKTWSPPAEELDAPLTLQDAQGLKDVLLTAFAYRQGLQELITGNPDKALSSLHEAASGLCPRPVLVQVYTALGSCHRKMGNPQRALLYLVAALKEGSAWGPPLLEASRLYQQLGDTTAELESLELLVEALNVPCSSKAPQFLIEVELLLPPPDLASPLHCGTQSQTKHILASRCLQTGRAGDAAEHYLDLLALLLDSSEPRFSPPPSPPGPCMPEVFLEAAVALIQAGRAQDALTLCEELLSRTSSLLPKMSRLWEDARKGTKELPYCPLWVSATHLLQGQAWVQLGAQKVAISEFSRCLELLFRATPEEKEQGAAFNCEQGCKSDAALQQLRAAALISRGLEWVASGQDTKALQDFLLSVQMCPGNRDTYFHLLQTLKRLDRRDEATALWWRLEAQTKGSHEDALWSLPLYLESYLSWIRPSDRDAFLEEFRTSLPKSCDL

SEQ ID NO: 14 - protein, 1833 aa, homo sapiens
  MISC_FEATURE: amino acid sequence of the SLX4 protein (corresponding to NP_115820.2)
MKLSVNEAQLGFYLGSLSHLSACPGIDPRSSEDQPESLKTGQMMDESDEDFKELCASFFQRVKKHGIKEVSGERKTQKAASNGTQIRSKLKRTKQTATKTKTLQGPAEKKPPSGSQAPRTKKQRVTKWQASEPAHSVNGEGGVLASAPDPPVLRETAQNTQTGNQQEPSPNLSREKTRENVPNSDSQPPPSCLTTAVPSPSKPRTAQLVLQRMQQFKRADPERLRHASEECSLEAAREENVPKDPQEEMMAGNVYGLGPPAPESDAAVALTLQQEFARVGASAHDDSLEEKGLFFCQICQKNLSAMNVTRREQHVNRCLDEAEKTLRPSVPQIPECPICGKPFLTLKSRTSHLKQCAVKMEVGPQLLLQAVRLQTAQPEGSSSPPMFSFSDHSRGLKRRGPTSKKEPRKRRKVDEAPSEDLLVAMALSRSEMEPGAAVPALRLESAFSERIRPEAENKSRKKKPPVSPPLLLVQDSETTGRQIEDRVALLLSEEVELSSTPPLPASRILKEGWERAGQCPPPPERKQSFLWEGSALTGAWAMEDFYTARLVPPLVPQRPAQGLMQEPVPPLVPPEHSELSERRSPALHGTPTAGCGSRGPSPSASQREHQALQDLVDLAREGLSASPWPGSGGLAGSEGTAGLDVVPGGLPLTGFVVPSQDKHPDRGGRTLLSLGLLVADFGAMVNNPHLSDVQFQTDSGEVLYAHKFVLYARCPLLIQYVNNEGFSAVEDGVLTQRVLLGDVSTEAARTFLHYLYTADTGLPPGLSSELSSLAHRFGVSELVHLCEQVPIATDSEGKPWEEKEAENCESRAENFQELLRSMWADEEEEAETLLKSKDHEEDQENVNEAEMEEIYEFAATQRKLLQEERAAGAGEDADWLEGGSPVSGQLLAGVQVQKQWDKVEEMEPLEPGRDEAATTWEKMGQCALPPPQGQHSGARGAEAPEQEAPEEALGHSSCSSPSRDCQAERKEGSLPHSDDAGDYEQLFSSTQGEISEPSQITSEPEEQSGAVRERGLEVSHRLAPWQASPPHPCRFLLGPPQGGSPRGSHHTSGSSLSTPRSRGGTSQVGSPTLLSPAVPSKQKRDRSILTLSKEPGHQKGKERRSVLECRNKGVLMFPEKSPSIDLTQSNPDHSSSRSQKSSSKLNEEDEVILLLDSDEELELEQTKMKSISSDPLEEKKALEISPRSCELFSIIDVDADQEPSQSPPRSEAVLQQEDEGALPENRGSLGRRGAPWLFCDRESSPSEASTTDTSWLVPATPLASRSRDCSSQTQISSLRSGLAVQAVTQHTPRASVGNREGNEVAQKFSVIRPQTPPPQTPSSCLTPVSPGTSDGRRQGHRSPSRPHPGGHPHSSPLAPHPISGDRAHFSRRFLKHSPPGPSFLNQTPAGEVVEVGDSDDEQEVASHQANRSPPLDSDPPIPIDDCCWHMEPLSPIPIDHWNLERTGPLSTSSPSRRMNEAADSRDCRSPGLLDTTPIRGSCTTQRKLQEKSSGAGSLGNSRPSFLNSALWDVWDGEEQRPPETPPPAQMPSAGGAQKPEGLETPKGANRKKNLPPKVPITPMPQYSIMETPVLKKELDRFGVRPLPKRQMVLKLKEIFQYTHQTLDSDSEDESQSSQPLLQAPHCQTLASQTYKPSRAGVHAQQEATTGPGAHRPKGPAKTKGPRHQRKHHESITPPSRSPTKEAPPGLNDDAQIPASQESVATSVDGSDSSLSSQSSSSCEFGAAFESAGEEEGEGEVSASQAAVQAADTDEALRCYIRSKPALYQKVLLYQPFELRELQAELRQNGLRVSSRRLLDFDTHCITFTTAATRREKLQGRRRQPRGKKKVERN

SEQ ID NO: 15 - protein, 1056 aa, homo sapiens
  MISC_FEATURE: amino acid sequence of the kinesin-related motor protein Eg5 (corresponding to NP_004514.2) (Note: the official symbol of the corresponding coding gene is KIF11)
MASQPNSSAKKKEEKGKNIQVVVRCRPFNLAERKASAHSIVECDPVRKEVSVRTGGLADKSSRKTYTFDMVFGASTKQIDVYRSVVCPILDEVIMGYNCTIFAYGQTGTGKTFTMEGERSPNEEYTWEEDPLAGIIPRTLHQIFEKLTDNGTEFSVKVSLLEIYNEELFDLLNPSSDVSERLQMFDDPRNKRGVIIKGLEEITVHNKDEVYQILEKGAAKRTTAATLMNAYSSRSHSVFSVTIHMKETTIDGEELVKIGKLNLVDLAGSENIGRSGAVDKRAREAGNINQSLLTLGRVITALVERTPHVPYRESKLTRILQDSLGGRTRTSIIATISPASLNLEETLSTLEYAHRAKNILNKPEVNQKLTKKALIKEYTEEIERLKRDLAAAREKNGVYISEENFRVMSGKLTVQEEQIVELIEKIGAVEEELNRVTELFMDNKNELDQCKSDLQNKTQELETTQKHLQETKLQLVKEEYITSALESTEEKLHDAASKLLNTVEETTKDVSGLHSKLDRKKAVDQHNAEAQDIFGKNLNSLFNNMEELIKDGSSKQKAMLEVHKTLFGNLLSSSVSALDTITTVALGSLTSIPENVSTHVSQIFNMILKEQSLAAESKTVLQELINVLKTDLLSSLEMILSPTVVSILKINSQLKHIFKTSLTVADKIEDQKKELDGFLSILCNNLHELQENTICSLVESQKQCGNLTEDLKTIKQTHSQELCKLMNLWTERFCALEEKCENIQKPLSSVQENIQQKSKDIVNKMTFHSQKFCADSDGFSQELRNFNQEGTKLVEESVKHSDKLNGNLEKISQETEQRCESLNTRTVYFSEQWVSSLNEREQELHNLLEVVSQCCEASSSDITEKSDGRKAAHEKQHNIFLDQMTIDEDKLIAQNLELNETIKIGLTKLNCFLEQDLKLDIPTGTTPQRKSYLYPSTLVRTEPREHLLDQLKRKQPELLMMLNCSENNKEETIPDVDVEEAVLGQYTEEPLSQEPSVDAGVDCSSIGGVPFFQHKKSHGKDKENRGINTLERSKVEETTEHLVTKSRLPLRAQINL

SEQ ID NO: 16 - protein, 205 aa, homo sapiens
  MISC_FEATURE: amino acid sequence of the MAD2A protein (corresponding to NP_002349.1). (Note: the official symbol of the corresponding coding gene is MAD2L1)
MALQLSREQGITLRGSAEIVAEFFSFGINSILYQRGIYPSETFTRVQKYGLTLLVTTDLELIKYLNNVVEQLKDWLYKCSVQKLVVVISNIESGEVLERWQFDIECDKTAKDDSAPREKSQKAIQDEIRSVIRQITATVTFLPLLEVSCSFDLLIYTDKDLVVPEKWEESGPQFITNSEEVRLRSFTTTIHKVNSMVAYKIPVND

SEQ ID NO: 17 - protein, 542 aa, homo sapiens
  MISC_FEATURE: amino acid sequence of the TRF2 (corresponding to NP_005643.2). (Note: the official symbol of the corresponding coding gene is TERF2)
MAAGAGTAGPASGPGVVRDPAASQPRKRPGREGGEGARRSDTMAGGGGSSDGSGRAAGRRASRSSGRARRGRHEPGLGGPAERGAGEARLEEAVNRWVLKFYFHEALRAFRGSRYGDFRQIRDIMQALLVRPLGKEHTVSRLLRVMQCLSRIEEGENLDCSFDMEAELTPLESAINVLEMIKTEFTLTEAVVESSRKLVKEAAVIICIKNKEFEKASKILKKHMSKDPTTQKLRNDLLNIIREKNLAHPVIQNFSYETFQQKMLRFLESHLDDAEPYLLTMAKKALKSESAASSTGKEDKQPAPGPVEKPPREPARQLRNPPTTIGMMTLKAAFKTLSGAQDSEAAFAKLDQKDLVLPTQALPASPALKNKRPRKDENESSAPADGEGGSELQPKNKRMTISRLVLEEDSQSTEPSAGLNSSQEAASAPPSKPTVLNQPLPGEKNPKVPKGKWNSSNGVEEKETWVEEDELFQVQAAPDEDSTTNITKKQKWTVEESEWVKAGVQKYGEGNWAAISKNYPFVNRTAVMIKDRWRTMKRLGMN